SEQUENCE LISTING (42 sequences)

SEQ ID NO: 1 - protein, 88 aa, Artificial Sequence
  Gp41.1 Int-N
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 2 - protein, 37 aa, Artificial Sequence
  Gp41.1 Int-C
MMLKKILKIEELDERELIDIEVSGNHLFYANDILTHN

SEQ ID NO: 3 - protein, 101 aa, Artificial Sequence
  Cfa Int-N
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 4 - protein, 36 aa, Artificial Sequence
  Cfa Int-C
MVKIISRKSLGTQNVYDIGVEKDHNFLLKNGLVASN

SEQ ID NO: 5 - protein, 104 aa, Artificial Sequence
  NRDJ-1 Int-N
CLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVA

SEQ ID NO: 6 - protein, 41 aa, Artificial Sequence
  NRDJ-1 Int-C
MIEAKTYIGKLKSRKIVSNEDTYDIQTSTHNFFANDILVHN

SEQ ID NO: 7 - protein, 101 aa, Artificial Sequence
  IMPDH-1 Int-N
CFVPGTLVNTENGLKKIEEIKVGDKVFSHTGKLQEVVDTLIFDRDEEIISINGIDCTKNHEFYVIDKENANRVNEDIHLFARWVVHAEELDMKKHLLIELE

SEQ ID NO: 8 - protein, 40 aa, Artificial Sequence
  IMPDH-1 Int-C
MKFKLKEITSIETKHYKGKVHDLTVNQDHSYNVRGTVVHN

SEQ ID NO: 9 - protein, 100 aa, Artificial Sequence
  Npu Int-N
CLSYETEILTVEYGLLPIGKIVEKRIECTVYSVDNNGNIYTQPVAQWHDRGEQEVFEYCLEDGSLIRATKDHKMTVDGQMLPIDEIFERELDLMRDNLPN

SEQ ID NO: 10 - protein, 36 aa, Artificial Sequence
  Npu Int-C
MIKIATRKYLGKQNVYDIGVERDHNFALKNGFIASN

SEQ ID NO: 11 - protein, 123 aa, Artificial Sequence
  Ssp Int-N
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 12 - protein, 36 aa, Artificial Sequence
  Ssp Int-C
MVKVIGRRSLGVQRIFDIGLPQDHNFLLANGAIAAN

SEQ ID NO: 13 - protein, 122 aa, Artificial Sequence
  Rma Int-N
CLAGDTLITLADGRRVPIRELVSQQNFSVWALNPQTYRLARVSRARVSRAFCTGIKPVYRLTTRLGRSIRATANHRFLTQGWKRVDELQPGDYLALPRRIPTASTPTLTEAELALLGHLIGD

SEQ ID NO: 14 - protein, 37 aa, Artificial Sequence
  Rma Int-C
MWDPIVSIEPDGVEEVFDLTVPGPHNFVADNIIAGNS

SEQ ID NO: 15 - protein, 118 aa, Artificial Sequence
  Ppu Int-N
CISKFSHIMWSHVSKPLFNFSIKKSHMHNGNKNIYQLLDQGEAFISRQDKKTTYKIRTNSEKYLELTSNHKILTLRGWQRCDQLLCNDMITTQIGFELSRKKKYLLNCIPFSLCNFET

SEQ ID NO: 16 - protein, 34 aa, Artificial Sequence
  Ppu Int-C
MLANINISNFQNVFDFAANPIPNFIANNIIVHNS

SEQ ID NO: 17 - protein, 88 aa, Artificial Sequence
  Gp41.8 Int-N
CLSLDTMVVTNGKAIEIRDVKVGDWLESECGPVQVTEVLPIIKQPVFEIVLKSGKKIRVSANHKFPTKDGLKTINSGLKVGDFLRSRA

SEQ ID NO: 18 - protein, 45 aa, Artificial Sequence
  Gp41.8 Int-C
MCEIFENEIDWDEIASIEYVGVEETIDINVTNDRLFFANGILTHN

SEQ ID NO: 19 - protein, 105 aa, Artificial Sequence
  NrdA-2 Int-N
CLTGDAKIDVLIDNIPISQISLEEVVNLFNEGKEYVLSYNIDTKEVEYKEISDAGLISESAEVLEIIDEETGQKIVCTPDHKVYTLNRGYVSAKDLKEDDELVFS

SEQ ID NO: 20 - protein, 35 aa, Artificial Sequence
  NrdA-2 Int-C
MGLKIIKRESKEPVFDITKVKDNSNFFANNILVHN

SEQ ID NO: 21 - DNA, 4527 bp, Artificial Sequence
  Translysin (T5 lys endolysin - HN - Pb5 RBP)
atgcatcatcatcatcatcacagcttcaaattcggcaaaaacagcgaaaaacagctggcaaccgttaaaccggaactgcagaaagttgcacgtcgtgcactggaactgagcccgtatgattttaccattgttcagggtattcgtaccgttgcacagagcgcacagaatattgcaaatggcaccagctttctgaaagatccgagcaaaagcaaacatattaccggtgatgcaattgatttcgcaccgtatattaacggcaaaatcgattggaatgatctggaagcattttgggcagtgaaaaaagcatttgaacaggcaggtaaagaactgggtattaaactgcgttttggtgcagattggaacgcaagcggtgattatcatgatgaaattaaacgtggcacctatgatggtggtcatgttgaactggttggtggaggcggtagcccgtttgtgaacaaacagttcaactataaagatccggtgaacggtgttgatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaaagctttggccacgatgttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccgaacatcgtctgtatggtattgccattaatccgaatcgtgtgttcaaagtgaataccaacgcctattatgaaatgagcggtctggaagtgagttttgaagaactgcgtacctttggtggtcatgatgccaaatttatcgatagcctgcaagaaaatgaatttcgcctgtactactataacaaattcaaagatgttgcgagcaccctgaataaagccaaaagcattattggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaacttctttaaggtgatcaacgccaaaaccttcctgaactttgataaagccgtgtttcgcattaacattgtgccggatgaaaactacaccatcaaagatggctttaatctgaagggtgcaaatctgtccaccaattttaacggtcagaacaccgaaattaacagccgtaattttacccgtctgaaaaactttaccggtctgttcgaattttacaaactgctgtgtgttcgtggcattatcccgtttaaagagaacctgtattttcagggtgcactgaatgatctgtgcatcaaagtgaataattgggacctgttttttagcccgagcgaagataactttaccaacgatctggataaagtggaagaaattaccgcagataccaatattgaagcagccgaagaaaacattagcctggatctgattcagcagtattatctgaccttcgattttgataacgagccggaaaatatcagcattgaaaatctgagcagcgatattattggtcagctggaaccgatgccgaatattgaacgttttccgaatggcaaaaaatacgagctggacaaatataccatgttccattatctgcgtgcccaagaatttgaacatggtgatagccgcattattctgaccaattcagcagaagaagcactgctgaaaccgaatgttgcatatacctttttcagcagcaaatatgtgaaaaaaatcaacaaagccgtcgaagcctttatgtttctgaattgggctgaagaactggtgtatgatttcaccgatgaaaccaatgaagttaccaccatggataaaattgccgacattaccattatcgtgccgtatattggtccggctctgaatattggcaatatgctgagcaaaggtgaatttgtggaagccattatctttaccggtgttgttgcaatgctggaatttatcccggaatatgcactgccggtttttggcacctttgcaattgttagctatatcgccaataaagttctgaccgttcagaccattaataacgcactgagcaaacgcaatgagaaatgggatgaagtgtataaatacaccgttaccaattggctggccaaagttaatacccagattgatctgatccgcgagaaaatgaaaaaagccctggaaaatcaggcagaagcaaccaaagcaattatcaactatcagtacaaccagtacaccgaggaagagaaaaacaacatcaacttcaacatcgatgacctgagcagcaaactgaatgaaagcattaatagcgccatgattaacatcaacaagtttctggatcagtgcagcgttagctatctgatgaatagcatgattccgtatgcagtgaaacgcctgaaagattttgatgcaagcgttcgtgatgtcctgctgaaatatatctatgataatcgtggcaccctggttctgcaggttgatcgtctgaaagatgaagttaataacaccctgagcgcagatattccgtttcagctgagtaaatatgtggacaacaaaaaactgctgagcacctttaccgagtacatcaaaaacggtggaggcggtagcatgagcttctttgcaggcaaactgaacaacaaaagcattctgagcctgcgtcgtggtagcggtggtgataccaatcagcatattaatccggatagccagaccatctttcatagcgatatgagccatgtgattatcaccgaaacacatagcaccggtctgcgtctggatcaaggtgccggtgattactattggagcgaaatgccgagccgtgttacccagctgcataataatgatccgaatcgtgttgttctgaccgaaattgaatttagtgatggtagccgtcatatgctgagcggtatgagcatgggtgttggtgcaaaagcatatggtattatcaatccgcagattatgagccaaggtggtctgaaaacccagattaccgcaagcgcagatctgagtctggatgttggctattttaacaccggtacaagcggcaccattccgcagaaactgcgtgatggcaccggttgccagcatatgtttggtgcatttagcggtcgtcgtggttttgcaagcagcgcaatgtatttaggtggtgcagcactgtataaaagcgcatggtcaggtagcggttatgttgttgcagatgcaggcaccctgaccattccgagcgattatgttcgtcatccgggtgcacgtaattttggttttaatgcaatctatgttcgtggtcgtagctgtaatcgtgttctgtatggtatggaaggtccgaattatacaaccggtggtgccgttcagggtgcgagcagcagcggtgcactgaattttacctataatccgagcaatccggaaagcccgaaatatagcgttggttttgcccgtgcagatccgaccaattatgcctattgggaaagtatgggcgatccgaatgatagcgcaaatggtccgattggtatttatagcgaacatctgggtatctacccgagcaaaattacctggtatgttaccaatctggtgtataatggtagcggctataacattgatggtggtctgtttaatggcaacgacattaaactgagtccgcgtgaattcattatcaaaggcgtgaatgtgaataataccagctggaagtttatcaacttcatcgagaaaaacttcaacgtgggtaatcgtgcagattttcgtgatgttggttgtaacctgagcaaagatagcccgagcaccggtattagcggtattgcaacctttggtctgccgaccaccgaaagcaataatgcaccgagcattaaaggtggtaatgttggtggcctgcatgcaaatgttgttagcatttataactttctgccgagcgcaagctggtatgtgagcagcaatccgcctaaaattggtaataactatggtgatgtgtggtccgagaatctgctgccgctgcgtctgttaggtggtagtggtagcaccattctgagtggtaatattgtgtttcaaggtaatggcagcgttcatgttggcaccgttggtctggat

SEQ ID NO: 22 - DNA, 1911 bp, Artificial Sequence
  Innolysin (T5 lys endolysin - linker - Pb5 RBP)
atgagcttcaaattcggcaaaaacagcgaaaaacagctggcaaccgttaaaccggaactgcagaaagttgcacgtcgtgcactggaactgagcccgtatgattttaccattgttcagggtattcgtaccgttgcacagagcgcacagaatattgcaaatggcaccagctttctgaaagatccgagcaaaagcaaacatattaccggtgatgcaattgatttcgcaccgtatattaacggcaaaatcgattggaatgatctggaagcattttgggcagtgaaaaaagcatttgaacaggcaggtaaagaactgggtattaaactgcgttttggtgcagattggaacgcaagcggtgattatcatgatgaaattaaacgtggcacctatgatggtggtcatgttgaactggttgcaggtgcaggcgcaggcatgagcttctttgcaggcaaactgaacaacaaaagcattctgagcctgcgtcgtggtagcggtggtgataccaatcagcatattaatccggatagccagaccatctttcatagcgatatgagccatgtgattatcaccgaaacacatagcaccggtctgcgtctggatcaaggtgccggtgattactattggagcgaaatgccgagccgtgttacccagctgcataataatgatccgaatcgtgttgttctgaccgaaattgaatttagtgatggtagccgtcatatgctgagcggtatgagcatgggtgttggtgcaaaagcatatggtattatcaatccgcagattatgagccaaggtggtctgaaaacccagattaccgcaagcgcagatctgagtctggatgttggctattttaacaccggtacaagcggcaccattccgcagaaactgcgtgatggcaccggttgccagcatatgtttggtgcatttagcggtcgtcgtggttttgcaagcagcgcaatgtatttaggtggtgcagcactgtataaaagcgcatggtcaggtagcggttatgttgttgcagatgcaggcaccctgaccattccgagcgattatgttcgtcatccgggtgcacgtaattttggttttaatgcaatctatgttcgtggtcgtagctgtaatcgtgttctgtatggtatggaaggtccgaattatacaaccggtggtgccgttcagggtgcgagcagcagcggtgcactgaattttacctataatccgagcaatccggaaagcccgaaatatagcgttggttttgcccgtgcagatccgaccaattatgcctattgggaaagtatgggcgatccgaatgatagcgcaaatggtccgattggtatttatagcgaacatctgggtatctacccgagcaaaattacctggtatgttaccaatctggtgtataatggtagcggctataacattgatggtggtctgtttaatggcaacgacattaaactgagtccgcgtgaattcattatcaaaggcgtgaatgtgaataataccagctggaagtttatcaacttcatcgagaaaaacttcaacgtgggtaatcgtgcagattttcgtgatgttggttgtaacctgagcaaagatagcccgagcaccggtattagcggtattgcaacctttggtctgccgaccaccgaaagcaataatgcaccgagcattaaaggtggtaatgttggtggcctgcatgcaaatgttgttagcatttataactttctgccgagcgcaagctggtatgtgagcagcaatccgcctaaaattggtaataactatggtgatgtgtggtccgagaatctgctgccgctgcgtctgttaggtggtagtggtagcaccattctgagtggtaatattgtgtttcaaggtaatggcagcgttcatgttggcaccgttggtctggatcatcatcatcatcatcac

SEQ ID NO: 23 - DNA, 738 bp, Artificial Sequence
  LysPA26-Gp41.1N
atgcgcaccaataacattgatgccatcaaagaacatgaaggtctgcgtctggttgcatatctggatagcgttggtgtttggaccattggttatggtgataccggtccggatgttgttaaaggtctgaccattaccaaagaagaagcagaaaaacgtctgcgtaaacgtctggtggaatttgaaggttatgtgaacacctatgttaaagtgccgctgaaacagcatcagtttgatgcactggttagcctggtttataacattggtccgaccaactttaaaacaagcaccctgctgaaaaaactgaacgcaggcgattatattggtgcagcagatcagtttctggtgtggaataaaggtcgtgttgatggtaaactggtggttattaaaggcctggcaaatcgtcgtgcaaaagaacgtaaacagttcctgggtgaaggtggaggcggtagcggttattgcctggatctgaaaacccaggttcagacaccgcagggtatgaaagaaatttcaaatattcaggtgggtgatctggttctgagcaataccggttataatgaagtgctgaatgtgttcccgaaaagcaagaaaaaaagctacaaaatcaccctggaagatggcaaagaaattatctgtagcgaagaacacctgtttccgacacagaccggtgaaatgaatattagcggtggtctgaaagaaggtatgtgcctgtatgttaaagaacatcatcatcatcatcac

SEQ ID NO: 24 - DNA, 963 bp, Artificial Sequence
  PB1_gp48-Gp41.1N
atgaagatcaccaaagatattctgattaccggcaccggttgtaccaccgatcgtgcaattaaatggctggatgatattcaggcagccatggataaatttcagattgaaagtccgcgtgcaattgccgcatatctggcaaatattggtgttgaaagcggtggtctggttagcctggttgaaaatctgaattatagcgcacaaggtctggccaatacctggcctcgtcgttatgcagttgatccgcgtgttcgtccgtatgttccgaatgcactggcaaatcgtctggcacgtaatccggttgccattgcaaataatgtttatgcagatcgcatgggtaatggttgtgaacaggatggtgatggttggaaatatcgtggtcgtggtctgattcagctgaccggtaaaagcaattatgcactgtttgccgaagatagcggtatggatgttctggaaaaaccggaactgctggaaacaccggcaggcgcaagcatgagcagcgcatggtttttttggcgtaatcgttgtattccgatggccgaaagcaataactttagcatggtggtgaaaaccattaacggtgcagcaccgaatgatgcaaatcatggtcagctgcgtattaaccgttatgttaaaaccgttgcagccattaatcagggcagcggtggaggcggtagcggttattgcctggatctgaaaacccaggttcagacaccgcagggtatgaaagaaatttcaaatattcaggtgggtgatctggttctgagcaataccggttataatgaagtgctgaatgtgttcccgaaaagcaagaaaaaaagctacaaaatcaccctggaagatggcaaagaaattatctgtagcgaagaacacctgtttccgacacagaccggtgaaatgaatattagcggtggtctgaaagaaggtatgtgcctgtatgttaaagaacatcatcatcatcatcac

SEQ ID NO: 25 - DNA, 3054 bp, Artificial Sequence
  Gp41.1C-iLCHN-CfaN
atgatgctgaaaaaaatcctgaaaatcgaggaactggatgaacgcgaactgattgatattgaagttagcggtaaccacctgttctatgccaatgatattctgacccataatagcagcagcccgtttgtgaacaaacagttcaactataaagatccggtgaacggtgttgatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaaagctttggccacgatgttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccgaacatcgtctgtatggtattgccattaatccgaatcgtgtgttcaaagtgaataccaacgcctattatgaaatgagcggtctggaagtgagttttgaagaactgcgtacctttggtggtcatgatgccaaatttatcgatagcctgcaagaaaatgaatttcgcctgtactactataacaaattcaaagatgttgcgagcaccctgaataaagccaaaagcattattggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaacttctttaaggtgatcaacgcgaaaacctttctgaactttgataaagccgtgtttcgcattaacattgtgccggatgaaaactacaccatcaaagatggctttaatctgaagggtgcaaatctgtccaccaattttaacggtcagaacaccgaaattaacagccgtaattttacccgtctgaaaaactttaccggtctgttcgaattttacaaactgctgtgtgttcgtggcattatcccgtttaaagagaacctgtattttcagggtgcactgaatgatctgtgcatcaaagtgaataattgggacctgttttttagcccgagcgaagataactttaccaacgatctggataaagtggaagaaattaccgcagataccaatattgaagcagccgaagaaaacattagcctggatctgattcagcagtattatctgaccttcgattttgataacgagccggaaaatatcagcattgaaaatctgagcagcgatattattggtcagctggaaccgatgccgaatattgaacgttttccgaatggcaaaaaatacgagctggacaaatataccatgttccattatctgcgtgcccaagaatttgaacatggtgatagccgcattattctgaccaattcagcagaagaagcactgctgaaaccgaatgttgcatatacctttttcagcagcaaatatgtgaaaaaaatcaacaaagccgtcgaagcctttatgtttctgaattgggctgaagaactggtgtatgatttcaccgatgaaaccaatgaagttaccaccatggataaaattgccgacattaccattatcgtgccgtatattggtccggctctgaatattggcaatatgctgagcaaaggtgaatttgtggaagccattatctttaccggtgttgttgcaatgctggaatttatcccggaatatgcactgccggtttttggcacctttgcaattgttagctatatcgccaataaagttctgaccgttcagaccattaataacgcactgagcaaacgcaatgagaaatgggatgaagtgtataaatacaccgttaccaattggctggccaaagttaatacccagattgatctgatccgcgagaaaatgaaaaaagccctggaaaatcaggcagaagcaaccaaagcaattatcaactatcagtacaaccagtacaccgaggaagagaaaaacaacatcaacttcaacatcgatgacctgagcagcaaactgaatgaaagcattaatagcgccatgattaacatcaacaagtttctggatcagtgcagcgttagctatctgatgaatagcatgattccgtatgcagtgaaacgcctgaaagattttgatgcaagcgttcgtgatgtcctgctgaaatatatctatgataatcgtggcaccctggttctgcaggttgatcgtctgaaagatgaagttaataacaccctgagcgcagatattccgtttcagctgagtaaatatgtggacaacaaaaaactgctgagcacctttaccgagtacatcaaaaacgccgaatattgcctgtcttacgacacagagattctgaccgttgaatatggattccttcctatcggtaagatcgtggaggaacggattgaatgcacagtctatacggtagataaaaatggctttgtgtatacacaacctattgctcagtggcataaccggggagaacaggaagttttcgaatactgcttagaagacggttcgattatccgtgcaacgaaagatcacaaatttatgacgaccgacggtcagatgttaccgattgatgagattttcgaacgggggttagacctgaaacaagttgatggtttgccgcaccaccaccaccaccac

SEQ ID NO: 26 - DNA, 1923 bp, Artificial Sequence
  CfaC-PRD1_04
atgcatcatcatcatcatcacgtcaagatcattagtcgtaagagtctgggcactcaaaacgtctacgatattggagtagaaaaagatcataattttttgctgaagaatgggctggtggcctctaactgcttcaacggtggaggcggtagcatggccaattttaacgttccgaaactgggtgtttttccggttgcagcagtttttgatattgataatgttccggaagatagcagcgcaaccggtagccgttggctgccgagcatttatcaaggtggtaattattggggtggtggtccgcaggcactgcatgcacaggttagcaattttgatagcagcaatcgtctgccgtataatccgcgtaccgaaaataatccggcaggtaattgtgcatttgcgtttaatccgtttggtcagtatattagcaatatcagcagcgcacagagcgttcatcgtcgtatttatggtattgatctgaatgatgaaccgctgtttagcccgaatgcagcaagcattaccaatggtggcaatccgaccatgagccaggataccggttatcataatattggtccgattaacaccgcctataaagccgaaatttttcgtccggttaatccgctgccgatgagcgataccgcaccggatcctgaaacactggaaccgggtcagaccgaaccgctgattaaaagtgatggtgtttatagcaatagcggcattgccagctttatctttgatcgtccggtgacagaaccgaatccgaattggcctccgctgcctccgcctgttattccgattatctatccgacaccggcattaggtattggtgcagcagcagcatatggttttggttatcaggttaccgtttatcgctgggaagaaattccggttgaatttattgcagatccggaaacctgtccggcacagccgaccaccgataaagttattattcgtaccaccgatctgaatccggaaggtagcccgtgtgcatatgaagcaggtattattctggttcgtcagaccagcaatccgatgaatgcagttgcaggtcgtctggttccgtatgttgaagatattgccgttgatatttttctgaccggcaaattctttaccctgaatccgcctctgcgtattaccaataactattttgcagatgacgaagtgaaagaaaacaccgttaccattggcaattataccaccacactgagcagcgcatattatgcagtgtataaaaccgatggctatggtggtgcaacctgttttattgcaagcggtggtgccggtattagcgcactggttcagctgcaggataatagcgttctggatgttctgtattatagcctgccgctgagcttaggtggtagcaaagcagcaattgatgaatgggttgcaaataactgtggtctgtttccgatgagtggtggtctggataaaaccacactgctggaaattccgcgtcgtcagctggaagcaattaatccgcaggatggtccgggtcagtatgacctgtttattctggatgatagcggtgcctatgcaagctttagcagctttattggttatccggaagcagcctattatgttgccggtgcagcaacctttatggatgttgaaaatccggatgagatcatctttattctgcgtaatggtgcaggttggtatgcatgtgaaattggtgatgcactgaaaatcgccgatgatgaatttgatagcgtcgattattttgcctatcgtggtggtgttatgtttattggtagcgcacgttataccgaaggtggtgatcctctgccgatcaaatatcgtgcaattattccgggtctgcctcgtggtcgtctgccacgtgttgttctggaatatcaggcagttggtatgagctttattccgtgtcagacccattgtttaggtaaaggtggcattatcagcaaggtg

SEQ ID NO: 27 - DNA, 714 bp, Artificial Sequence
  T5Lys-Gp41.1N
atgagcttcaaattcggcaaaaacagcgaaaaacagctggcaaccgttaaaccggaactgcagaaagttgcacgtcgtgcactggaactgagcccgtatgattttaccattgttcagggtattcgtaccgttgcacagagcgcacagaatattgcaaatggcaccagctttctgaaagatccgagcaaaagcaaacatattaccggtgatgcaattgatttcgcaccgtatattaacggcaaaatcgattggaatgatctggaagcattttgggcagtgaaaaaagcatttgaacaggcaggtaaagaactgggtattaaactgcgttttggtgcagattggaacgcaagcggtgattatcatgatgaaattaaacgtggcacctatgatggtggtcatgttgaactggttggtggaggcggtagcggttattgcctggatctgaaaacccaggttcagacaccgcagggtatgaaagaaatttcaaatattcaggtgggtgatctggttctgagcaataccggttataatgaagtgctgaatgtgttcccgaaaagcaagaaaaaaagctacaaaatcaccctggaagatggcaaagaaattatctgtagcgaagaacacctgtttccgacacagaccggtgaaatgaatattagcggtggtctgaaagaaggtatgtgcctgtatgttaaagaacatcatcatcatcatcac

SEQ ID NO: 28 - DNA, 1905 bp, Artificial Sequence
  CfaC-T5likechee24
atgcatcatcatcatcatcacgtcaagatcattagtcgtaagagtctgggcactcaaaacgtctacgatattggagtagaaaaagatcataattttttgctgaagaatgggctggtggcctctaactgcttcaacggtggaggcggtagcatgggtttctttgccggtaaatatagtgatggtaaaaccgttctgagcctgaataccgaaagcggtggtgatattaatcgtcattatagcccgaatgccaacagcatttttcatagcgatatgccgtttgttctggttgatggcacctatgaagttggtctgggtgatgcaggtaatggtttttttgtttgtcagatgcctccggatatcgtgaacattaaaagcaatgatccgggtcgtgttattctgaccgcaattgaaattaatggcacccatcgtgcatttctgaatggtacacagagcaaagttggtcagaccattgttgcaacccaggcagatccgtttcgtagctttgcaagcgttagccagaccagcggttttgcatttggtaatagcctggcaagcggcacctataactataatccgagcctgggtcatgaagaaagcattagccgtagcggcaccggtggtacaaccctgcatagcacctatcatggtattgttcgtcctggtgcgggtgcaccggttggtattaccgttgcagaagcatttgcacagctgggttttccgaccaatagcagcaccgttccgattgatggtaataatccgtattattgggatcctggttggatgagtccgctgggtgcagcacatcgtggtcatgattggttttatgtttgcaatagcaacattcgcggttatggtggtgttcgtcagggtctgcctggtaatgttaataccatgtatcatgatggtggcaatcgttttgtttgccgtggtagcaccaccaatctggcaaatcagagcggtaatccgaccgttattcaggattggtacaatattaccccgaccaaagttatttggtatgtgctgaatctgcgctatagcaatggtggtatgagcattagtggtaatccgtttaccggtagcgatattctgattagcccgagcaactttattatcaaaggtgttagcctgccgaacaccggctataaattcattaatcagaacgcctttggcaacctgggttatcgtccggatatggaatatattggcaataatgcagcatacacgggtgtttttggtgataccaccgcacgttgtgaactggttggtagcagtaatggtggtctgtggtcaccggttgattatggtggcgcaaaaagccagattagcatttatcgttttggtgtgggtaaacagtggtatgtgaatagcaatgataacaccattggcaatgaacatggtgttgtttggggtccgagcgcagttccgctgcgtctgtttggtggtaatgtgggtagcagctatatgggtgatgatattacaccgagctatcctggtacaggtgatcgttatgttggtctgagcacaattggcctgggtattcctggcggtaatgcaaccgtgattctgaccaccgaagttatttcaggtaatctgaattgtgcgggtgttccggcaaatacctggaataatggtgtttttcaggttcagggtcgtcgtgcatatagctattcaggtggtgatgcaatctttcatcagattctgacactgccggttggttatctggttccgtttcataccacaagcgcatttcgttataccccgaataatgcactgagccgtaatagctttatctataccattaagaatcttggcaacggcaatgttgaactgggtgttgttatgcatgttagtctgggtagcgcagtttttctgcctcgtctgcgtgttaccgttcagcgtctgacc

SEQ ID NO: 29 - protein, 425 aa, Artificial Sequence
  HN
MALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKN

SEQ ID NO: 30 - protein, 438 aa, Artificial Sequence
  iLC
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINAKTFLNFDKAVFRINIVPDENYTIKDGFNLKGANLSTNFNGQNTEINSRNFTRLKNFTGLFEFYKLLCVRGIIPFK

SEQ ID NO: 31 - protein, 869 aa, Artificial Sequence
  iLCHN
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINAKTFLNFDKAVFRINIVPDENYTIKDGFNLKGANLSTNFNGQNTEINSRNFTRLKNFTGLFEFYKLLCVRGIIPFKENLYFQGALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKN

SEQ ID NO: 32 - protein, 116 aa, Artificial Sequence
  Spy001 Catcher
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 33 - protein, 13 aa, Artificial Sequence
  Spy001 Tag
AHIVMVDAYKPTK

SEQ ID NO: 34 - protein, 120 aa, Artificial Sequence
  Spy002 Catcher
AMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 35 - protein, 14 aa, Artificial Sequence
  Spy002 Tag
VPTIVMVDAYKRYK

SEQ ID NO: 36 - protein, 113 aa, Artificial Sequence
  Spy003 Catcher
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 37 - protein, 425 aa, Artificial Sequence; identical to SEQ ID NO: 29
  Spy003 Tag

SEQ ID NO: 38 - protein, 416 aa, Artificial Sequence
  HN
TLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYF

SEQ ID NO: 39 - protein, 421 aa, Artificial Sequence
  HN
KSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKF

SEQ ID NO: 40 - protein, 449 aa, Artificial Sequence
  iLC
PITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGQSLDQGG

SEQ ID NO: 41 - protein, 412 aa, Artificial Sequence
  iLC
PKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHQLIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTQFDLRTKFQVKCRQTYIGQYKYFKLSNLLNDSIYNISEGYNINNLKVNFRGQNANLNPRIITPITGRGLVKKIIRFCK

SEQ ID NO: 42 - protein, 1033 aa, Artificial Sequence
  Gp41.1C-iLCHN-SpyC003
MHHHHHHMMLKKILKIEELDERELIDIEVSGNHLFYANDILTHNSSSPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINAKTFLNFDKAVFRINIVPDENYTIKDGFNLKGANLSTNFNGQNTEINSRNFTRLKNFTGLFEFYKLLCVRGIIPFKENLYFQGALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNGGGGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

(19) United States
(12) Patent Application Publication
(10) Pub. No.: US 2024/0091322 A1
(43) Pub. Date: Mar. 21, 2024

(54) PROTEIN COMPLEX INCLUDING BOTULINUM TOXIN TRANSLOCATION DOMAIN AND ENDOLYSIN AND ANTIBACTERIAL COMPOSITION INCLUDING SAME

(71) Applicant: MVRIX Co., Ltd., Hwaseong-si, Gyeonggi-do (KR)

(72) Inventors: Dae Hyuk KWEON, Seoul (KR); Wonbeom PARK, Suwon-si (KR); Jihwan CHUN, Seoul (KR)

(73) Assignee: MVRIX Co., Ltd., Hwaseong-si, Gyeonggi-do (KR)

(21) Appl. No.: 18/287,223

(22) PCT Filed: Apr. 18, 2022
(86) PCT No.: PCT/KR2022/005511

§ 371 Date: Oct. 17, 2023

(30) Foreign Application Priority Data
Apr. 19, 2021 (KR) 10-2021-0050767

(51) Int. Cl. A61K 38/47; A61P 31/04; C12N 9/36; C12N 9/52

(52) U.S. Cl. CPC A61K 38/47; A61P 31/04; C12N 9/2462; C12N 9/52; C12Y 302/01017; C12Y 304/24069; C07K 2319/034; C07K 2319/92

(57) ABSTRACT

Provided is a protein complex including a botulinum toxin translocation domain and endolysin. When used, the protein complex including a botulinum translocation domain and endolysin according to the present invention exhibits an antibacterial effect and thus can be used as an antibacterial composition or an antibiotic.

### TECHNICAL FIELD

The present invention relates to a protein complex including a botulinum toxin translocation domain and endolysin.

### BACKGROUND ART

Infections with multidrug-resistant bacteria that are resistant to various antibiotics are causing serious medical problems in South Korea and other countries around the world. In South Korea, in accordance with the enforcement of the Infectious Disease Prevention and Control Act in December 2010, six types of antibiotic-resistant bacteria (VRSA (vancomycin-resistant Staphylococcus aureus), VRE (vancomycin-resistant Enterococcus), MRSA (methicillin-resistant Staphylococcus aureus), MRPA (multidrug-resistant Pseudomonas aeruginosa), MRAB (multidrug-resistant Acinetobacter baumannii), CRE (carbapenem-resistant Enterobacteriaceae) are designated and managed as surveillance targets. The U.S. Centers for Disease Control and Prevention has pointed out the seriousness of medical problems caused by hospital-derived six multidrug-resistant bacteria (Enterococcus faecium, Staphylococcus aureus, Klebsiella pneumoniae, Acinetobacter baumannii, Pseudomonas aeruginosa, and Enterobacter spp. ESKAPE) and ESBL (extended spectrum β-lactamase) strains and various multi-drug resistant bacteria such as carbapenem-resistant bacteria. Hospital-acquired infections caused by multidrug-resistant bacteria are reported to occur more frequently, especially in seriously ill patients, and treatment thereof is becoming increasingly difficult as they acquire more powerful resistance to antibiotics.

Bacteria are classified into Gram-positive and Gram-negative bacteria based on differences in Gram staining patterns depending on the cell wall structure and the cell wall structure thereof is as follows. Gram-positive bacteria consists of an inner membrane and peptidoglycan, and Gram-negative bacteria consists of an inner membrane, peptidoglycan, and an outer membrane. Thereamong, the cell outer membrane of Gram-negative bacteria is known to block various substances, including antibacterial substances, from being delivered into the cells.

Therefore, there is a need to develop antibacterial substances to effectively deliver target proteins into target strains.

### DISCLOSURE

### Technical Problem

The present inventors made extensive efforts to research and develop antibacterial substances to effectively deliver target proteins into target strains. As a result, the present inventors identified that the target proteins can be effectively delivered into the strains using a botulinum toxin translocation domain. Based thereon, the present invention has been completed.

Accordingly, it is one object of the present invention to provide a protein complex containing a botulinum toxin translocation domain and endolysin.

It is another object of the present invention to provide a recombinant vector containing a polynucleotide encoding the protein complex.

It is another object of the present invention to provide a host cell transformed with the recombinant vector containing the polynucleotide encoding the protein complex.

It is another object of the present invention to provide a method for producing a protein complex including mixing protein fragments and linking the mixed protein fragments.

It is another object of the present invention to provide an antibacterial composition containing the protein complex.

It is another object of the present invention to provide a method for screening a protein complex including reacting the protein complex with a target strain and determining whether or not the target strain exhibits antibacterial activity.

### Technical Solution

In accordance with one aspect of the present invention, the above and other objects can be accomplished by the provision of a protein complex containing a botulinum toxin translocation domain and endolysin.

In an embodiment of the present invention, the botulinum toxin translocation domain may include at least one amino acid sequence selected from SEQ ID NOS: 29, 38, and 39.

In one embodiment of the present invention, the endolysin may be selected from the group consisting of lys, LysPA26, PB1_gp48, LysAB2_P3, PlyF307, AcLys, PlyPA03, PlyPA91, Abtn-4, WCHABP1_gp01, WCHABP12_gp19, gh-1p12, B3ORF25, phi-13Sp4, phi-6S_4, KP27_166, KP13_gp066, BI057_gp221, LPSE_00024, STP4a_120, Lys68, SPN1S_0028 and P22gp66.

In one embodiment of the present invention, the protein complex may further contain a receptor-binding protein. The receptor-binding protein may be, for example, selected from the group consisting of PRD1_04, P1301_0153, P24_0149, Pb5, AbTJ_gp52, AbTJ_gp53, phiAB6_gp40, S, A318_gp060, rv5_gp030, rv5_gp033, PaoP5_075, BH773_gp153, AU075_gp145, CPT_Sugarland_191, JIPhKp127_0170, AmPhEK80_0178, P22_gp19, DET7_207, HWD08_gp154, BI021_gp088, HWD21_gp023, HWC41_gp146, HOS12_gp017, HOU44_gp075, I133_gp019, HWC50_gp066, and HOS34_gp106.

In one embodiment of the present invention, the protein complex may contain the endolysin, the botulinum toxin translocation domain, and the receptor-binding protein disposed in this order.

In accordance with another aspect of the present invention, provided is a recombinant vector containing a polynucleotide encoding the protein complex.

In accordance with another aspect of the present invention, provided is a host cell transformed with the recombinant vector containing the polynucleotide encoding the protein complex.

In one embodiment of the present invention, the polynucleotide may include a nucleic acid sequence represented by SEQ ID NO: 21.

In accordance with another aspect of the present invention, provided is a method of preparing a protein complex including (a) mixing a first protein fragment containing a botulinum toxin translocation domain with a second protein fragment containing endolysin, and (b) linking the mixed protein fragments to each other.

In one embodiment of the present invention, the method may further include mixing a third protein fragment containing a receptor-binding protein in the step (a) of mixing the first protein fragment containing the botulinum toxin translocation domain with the second protein fragment containing endolysin.

In one embodiment of the present invention, the protein fragment may contain intein.

In one embodiment of the present invention, the step (b) of linking the protein fragments may be performed by a protein trans-splicing reaction.

In one embodiment of the present invention, the protein fragment may contain SpyTag and Spycatcher.

In accordance with another aspect of the present invention, provided is an antibacterial composition containing the protein complex.

In accordance with another aspect of the present invention, provided is a method for screening an antibacterial protein complex including reacting the protein complex with a target strain, and determining whether or not the protein complex exhibits antibacterial activity against the target strain.

### Advantageous Effects

The aspects and advantages of the present invention are summarized as follows:

(a) The present invention provides a protein complex containing a botulinum toxin translocation domain and endolysin. (b) The present invention provides a recombinant vector containing a polynucleotide encoding the protein complex and a host cell transformed with the recombinant vector. (c) The present invention provides a method for producing a protein complex including mixing and linking protein fragments. (d) The present invention provides an antibacterial composition containing the protein complex. (e) The protein complex containing the botulinum toxin translocation domain and endolysin of the present invention provides an antibacterial effect and can thus be used as an antibacterial composition or antibiotic.

Further, the present invention provides a method for screening a protein complex including reacting the protein complex with a target strain and determining whether or not the protein complex exhibits antibacterial activity.

### DESCRIPTION OF DRAWINGS

FIG. 1 illustrates a vector for producing full-length translysin.

FIG. 2 illustrates the result of detection of translysin by SDS gel electrophoresis.

FIG. 3 illustrates the result of detection of innolysin by SDS gel electrophoresis.

FIG. 4 shows lytic effects depending on the presence or absence of intracellular expression of translysin and innolysin, as detected from the turbidity of a culture solution.

FIG. 5 is a graph showing growth patterns depending on the presence or absence of intracellular expression of translysin, innolysin, and endolysin, as detected from turbidity of the culture medium.

FIG. 6(A) shows four protein fragments (LysPA26-Gp41.1N, PB1_gp48-Gp41.1N, Gp41.1C-iLCH_{N}-CfaN, CfaC-PRD1_04) used to produce translysin. FIG. 6(B) shows three protein fragments (LysPA26-Gp41.1N, Gp41.1C-iLCH_{N}-Spy^{C}003, Spy^{T}003-PRD1_04) used to produce translysin.

FIG. 7 shows the result of trans-splicing of PB1_gp48 and iLCH_{N} over time.

FIG. 8 shows the result of trans-splicing of LysPA26 and iLCH_{N} over time.

FIG. 9 shows the result of trans-splicing of iLCH_{N} and PRD1 over time.

FIG. 10 shows the result of trans-splicing of PB1_gp48, iLCH_{N} and PRD1 over time.

FIG. 11 shows the results of trans-splicing and bioconjugation of LysPA26, iLCH_{N}, and PRD1 over time.

FIG. 12 is a schematic diagram illustrating a method of performing a spotting assay.

FIG. 13 shows the lytic activity of the LysPA26-Gp41.1N fragment against dead cells, detected by spotting assay.

FIG. 14 shows the lytic activity of the PB1_gp48-Gp41.1N fragment against dead cells, detected by spotting assay.

FIG. 15 shows the lytic activity of translysin (LysPA26-iLCH_{N}-PRD1) and endolysin fragments against live bacteria, detected by spotting assay.

FIG. 16 shows the lytic activity of translysin (PB1_gp48-iLCH_{N}-PRD1) and endolysin fragments against live bacteria, detected by spotting assay.

FIG. 17 shows the lytic activity of translysin (PB1_gp48-iLCH_{N}-PRD1) and endolysin fragments, and innolysin against live bacteria, detected by spotting assay.

FIG. 18 shows the lytic activity of translysin (LysPA26-iLCH_{N}-PRD1) and endolysin fragments, and innolysin against live bacteria, detected by spotting assay.

FIG. 19 is an electron microscope image showing viable cells depending on the translysin treatment and time.

FIG. 20 shows the lytic activity of translysin and innolysin detected through CFU reduction assay.

FIG. 21 shows a schematic diagram illustrating screening of a translysin library against target strains.

FIG. 22 shows the lysis patterns of viable cells of target strains as a function of concentration of each translysin.

FIG. 23 is a schematic diagram illustrating the translysin structure of the present invention.

FIG. 24 is a schematic diagram illustrating the mechanism of action of translysin of the present invention.

FIG. 25 is a schematic diagram illustrating a method for synthesizing translysin using intein.

FIG. 26 is a schematic diagram illustrating a translysin synthesis method using intein and SpyTag and Spycatcher.

### BEST MODE

In one aspect, the present invention is directed to a protein complex containing a botulinum toxin translocation domain and endolysin.

As used herein, the term “protein complex” may be used interchangeably with “translysin.”

As used herein, the term “botulinum toxin translocation domain” refers to a protein that has the function of delivering light chains of botulinum toxin into nerve cells, and the delivery process occurs based on the mechanism in which when the botulinum toxin is introduced into the cells through endocytosis, the inside of the endosome becomes acidic, causing the translocation domain to have enzymatic activity, resulting in the process of transferring light chains from the endosome to the cytoplasm. As used herein, the term “botulinum toxin translocation domain” may also be referred to as “botulinum toxin translocation domain H_{N}”, “translocation domain”, “H_{N}”, or the like. In the present invention, the “botulinum toxin translocation domain” serves to allow the protein complex of the present invention to pass through the cell membrane (outer cell membrane). As a result, the protein complex of the present invention can access the peptidoglycan that exists between the outer and inner cell membranes.

In one embodiment of the present invention, the botulinum toxin translocation domain may include at least one amino acid sequence selected from SEQ ID NOS: 29, 38, and 39.

As used herein, the expression that “polynucleotide” (that may be used interchangeably with “gene”) or polypeptide (that may be used interchangeably with “protein”) “includes a specific nucleic acid sequence or amino acid sequence” or “includes a specific nucleic acid sequence or amino acid sequence.” may mean that the polynucleotide or polypeptide essentially includes the specific nucleic acid sequence or amino acid sequence, and may be interpreted as including a “substantially identical sequence” including a mutated (deleted, substituted, modified, and/or added) specific nucleic acid sequence or amino acid sequence (or not excluding the mutation) while maintaining the original function and/or desired function of the polynucleotide or polypeptide. For example, the expression that a polynucleotide or polypeptide “includes a specific nucleic acid sequence or amino acid sequence” or “includes or is represented by a specific nucleic acid sequence or amino acid sequence” means that the polynucleotide or polypeptide (i) essentially includes a sequence or amino acid sequence, or (ii) includes an amino acid sequence having an identity of at least 96%, at least 97%, at least 98%, at least 99%, at least 99.5%, or at least 99.9% to the specific nucleic acid or amino acid sequence and maintains original and/or desired functions thereof.

In one embodiment of the present invention, the protein complex may further contain a botulinum toxin light chain (LC). The addition of the botulinum toxin light chain (LC) may facilitate expression of the botulinum toxin translocation domain in a soluble form. At this time, it is preferable to use the botulinum toxin light chain in an inactivated state. According to an embodiment of the present invention, the botulinum toxin light chain may be obtained in an inactive form by modifying a zinc-binding sequence.

In one embodiment of the present invention, the inactivated botulinum toxin light chain (iLC) may include at least one selected from amino acid sequences represented by SEQ ID NOS: 30, 40, and 41 and the like.

In one embodiment of the present invention, the botulinum toxin translocation domain may be linked to the inactivated botulinum toxin light chain (iLC).

In one embodiment of the present invention, the polypeptide including the botulinum toxin translocation domain and the inactivated botulinum toxin light chain linked to each other may include an amino acid sequence of SEQ ID NO: 31.

As used herein, the term “endolysin” refers to a protein expressed in a virus that causes infection with bacteria as a host, and the endolysin exhibits the activity of lysing the cell wall of the host bacteria. Since endolysin cannot penetrate the outer cell membrane by itself, only the lytic activity of endolysin against Gram-positive bacteria is known. In addition, endolysin is not cytotoxic to eukaryotes including humans because it uses bacterial peptidoglycan as a substrate, and no cases of bacteria resistant to endolysin have been reported.

In the present invention, the protein complex of the present invention, which passes through the outer cell membrane and accesses peptidoglycan through the “botulinum toxin translocation domain” decomposes peptidoglycan through the endolysin contained therein and thus exhibits antibacterial activity.

In one embodiment of the present invention, the endolysin is selected from the group consisting of lys, LysPA26, PB1_gp48, LysAB2_P3, PlyF307, AcLys, PlyPA03, PlyPA91, Abtn-4, WCHABP1_gp01, WCHABP12_gp19, gh-1p12, B3ORF25, phi-13Sp4, phi-6S_4, KP27_166, KP13_gp066, BI057_gp221, LPSE_00024, STP4a_120, Lys68, SPN1S_0028 and P22gp66. However, any endolysin may be used in the present invention so long as it exhibit lytic activity and the endolysin is not necessarily limited to a specific type.

In one embodiment of the present invention, the protein complex may further contain a receptor-binding protein.

As used herein, the term “receptor-binding protein” refers to a structural protein involved in the process of recognizing and attaching to a specific receptor on the cell wall when a bacteriophage infects a host cell. The receptor-binding protein plays a role in maintaining stable binding while the genetic material of the bacteriophage is transferred into the host cell during the infection process and is known to be capable of binding to both gram-negative and gram-positive bacteria. Therefore, the present invention increases the contact of the protein complex with the strains and protein transfer efficiency using the characteristics of the receptor-binding protein. In other words, the present invention includes a “receptor-binding protein” and thus the protein complex of the present invention can be more easily and strongly bound to the target microorganism to be killed.

In one embodiment of the present invention, the receptor-binding protein may be selected from the group consisting of PRD1_04, P1301_0153, P24_0149, Pb5, AbTJ_gp52, AbTJ_gp53, phiAB6_gp40, S, A318_gp060, rv5_gp030, rv5_gp033, PaoP5_075, BH773_gp153, AU075_gp145, CPT_Sugarland_191, JIPhKp127_0170, AmPhEK80_0178, P22_gp19, DET7_207, HWD08_gp154, BI021_gp088, HWD21_gp023, HWC41_gp146, HOS12_gp017, HOU44_gp075, I133_gp019, HWC50_gp066 and HOS34_gp106. Any receptor-binding protein may be used so long as it can interact with the target strain and the receptor-binding protein is not necessarily limited to a specific type.

In one embodiment of the present invention, the protein complex has a configuration in which the endolysin, the botulinum toxin translocation domain, and the receptor-binding protein are disposed in this order.

In one embodiment of the present invention, the protein complex preferably has a configuration in which the endolysin, the botulinum toxin translocation domain, and the receptor-binding protein are disposed in this order from the amino terminus to the carboxyl terminus.

In one embodiment of the present invention, the protein complex has a configuration in which the endolysin, the botulinum toxin light chain, the botulinum toxin translocation domain, and the receptor-binding protein are disposed in this order from the amino terminus to the carboxyl terminus.

However, the order of the configuration of the protein complex described above is provided only as an example and thus the order of configuration is not limited thereto.

In one embodiment of the present invention, preferably, the protein complex has a configuration in which endolysin binds to the amino terminus of the botulinum toxin translocation domain and the receptor-binding protein binds to the carboxyl terminus of the botulinum toxin translocation domain.

In another aspect, the present invention is directed to a recombinant vector containing a polynucleotide encoding the protein complex.

As used herein, the term “vector” means a nucleic acid that includes a competent nucleotide sequence that is inserted into a host cell and recombines with and integrates into the host cell genome, or that replicates spontaneously as an episome. These vectors include linear nucleic acids, plasmids, phagemids, cosmids, RNA vectors, viral vectors and the like.

In another aspect, the present invention is directed to a host cell transformed with the recombinant vector containing the polynucleotide encoding the protein complex. The polynucleotide may include a nucleic acid sequence represented by SEQ ID NO: 21.

Host cells include cells that have been transfected, transformed, or infected with a recombinant vector or polynucleotide of the present invention, either in vivo or in vitro. The host cell containing the recombinant vector of the present invention is a recombinant host cell, a recombinant cell, a recombinant microorganism, or a mutant microorganism.

As used herein, the term “transformation” means a phenomenon in which DNA that is introduced into a host can be replicated with an extrachromosomal factor or by insertion into the chromosome.

As used herein, the term “encoded by” or “encoding” means that a polynucleotide expresses a polypeptide sequence.

In accordance with another aspect of the present invention, provided is a method of preparing a protein complex including mixing a first protein fragment containing a botulinum toxin translocation domain with a second protein fragment containing endolysin (a), and linking the mixed protein fragments to each other (b).

In one embodiment of the present invention, the method may further include mixing a third protein fragment containing a receptor-binding protein in the step (a) of mixing the first protein fragment containing the botulinum toxin translocation domain with the second protein fragment containing endolysin.

In one embodiment of the present invention, in the step of mixing (a), the first protein fragment containing the botulinum toxin translocation domain, the second protein fragment containing endolysin, and the third protein fragment containing the receptor-binding protein are mixed at a molar ratio of 1-5:1-5:1-5. According to an embodiment of the present invention, the mixing is preferably performed by mixing the first protein fragment, the second protein fragment, and the third protein fragment at a molar ratio of 1:1:1.

In the present invention, there is no limitation as to the order of mixing the protein fragments in the method for producing the protein complex. For example, the protein complex may be produced by mixing the first protein fragment containing the botulinum toxin translocation domain with the third protein fragment containing the receptor-binding protein, performing reaction, mixing the resulting product with the second protein fragment containing endolysin, and then performing reaction.

The botulinum toxin translocation domain, endolysin, and receptor-binding protein of the present invention are incorporated in the protein complex of one embodiment of the present invention and thus duplicate description is omitted to avoid excessive redundancy in the present specification.

In one embodiment of the present invention, the protein fragments, that is, the first protein fragment, the second protein fragment, and the third protein fragment, may include an intein. The intein is responsible for the reaction in which the two fragments of the split intein, Int^{N} and Int^{C}, spontaneously form a peptide bond and bind the extein linked to each fragment. The intein bound in this process is separated and removed from the newly bound extein. The protein binding reaction by inteins is referred to as “protein trans-splicing”.

In one embodiment of the present invention, the first protein fragment includes different types of inteins, namely, IntA^{C} and IntB^{N}, at both ends of the botulinum toxin translocation domain, the second protein fragment includes IntA^{N} and endolysin, and the third protein fragment includes receptor-binding protein and IntB^{C}. The Int^{A} and Int^{B} mean that they are different types of inteins.

In one embodiment of the present invention, the botulinum toxin translocation domain, endolysin, receptor-binding protein, and intein of the protein fragment may be linked by a linker.

As used herein, the term “linker” refers to a peptide inserted between two proteins, which are linked to produce another protein, in order to increase the structural flexibility of the proteins or enhance the activity of each protein. Any linker may be used without limitation as long as it does not inhibit the activity of each protein to be fused and does not cause an unnecessary immune response. The linker may be selected from the group consisting of a flexible amino acid linker, an inflexible linker, a cleavable amino acid linker, and a compound linker.

In one embodiment of the present invention, the intein may be selected from the group consisting of Gp41.1, Cfa, NRDJ-1, IMPDH-1, Npu, Ssp, Rma, Ppu, Gp41.8, and NrdA-2.

In one embodiment of the present invention, the step of linking the protein fragments may be performed by a protein trans-splicing reaction.

In one embodiment of the present invention, the protein fragment may include SpyTag and Spycatcher. SpyTag and SpyCatcher are used for irreversible conjugation of recombinant proteins. SpyTag and SpyCatcher spontaneously react with each other to form an intermolecular isopeptide bond therebetween and thereby enable bioconjugation between two recombinant proteins. According to the present invention, the SpyCatcher is present at the C terminus of the botulinum toxin translocation domain, the SpyTag is present in the receptor-binding protein, and the efficiency of binding between receptor-binding protein and the botulinum toxin translocation domain can be improved through the isopeptide bond of the SpyCatcher and SpyTag.

In one embodiment of the present invention, the step of linking the protein fragments may be performed by bioconjugation. Bioconjugation is a chemical reaction that links two biomolecules to each other, is generally a protein-protein conjugation, and serves as a key strategy for linking biomolecules to other substrates. Examples of bioconjugation include coupling of lysine, cysteine, and tyrosine amino acid residues, modification of tryptophan amino acid residues, and modification of the N- and C-termini. In the present invention, as described above, the binding efficiency between the botulinum toxin translocation domain and the receptor-binding protein is increased through the isopeptide bond between the SpyCatcher and the SpyTag.

In another aspect, the present invention is directed to an antibacterial composition containing the protein complex. At this time, the pH of the antibacterial composition is preferably adjusted to 7.4, and the antibacterial composition is not limited to any conditions known in the art. The antibacterial composition of the present invention exhibits antibacterial activity against Gram-positive and Gram-negative bacteria.

The gram-positive bacteria may be Gram-positive bacteria including Staphylococcus, Listeria, Streptococcus, Corynebacterium, Lactobacillus, Clostridium, Enterococcus, Erysipelothrix, and Bacillus, and may include all Gram-positive bacteria known in the art.

The gram-negative bacteria may be Gram-negative bacteria including Escherichia, Pseudomonas, Salmonella, Leptospira, Klebsiella, Helicobacter, and Enterobacter, and may include all Gram-negative bacteria known in the art.

The antibacterial composition of the present invention exhibits antibacterial activity against the following pathogens, but is not limited thereto:

Acinetobacter baumannii, Actinomyces sp. (for example, Actinomyces israelii and Actinomyces naeslundii, Aeromonas sp. (for example, Aeromonas hydrophila, Aeromonas veronii biovar sobria (Aeromonas sobria)) and Aeromonas caviae, Anaplasma phagocytophilum, Alcaligenes xylosoxidans, Actinobacillus actinomycetemcomitans, Bacillus sp. (for example, Bacillus anthracis, Bacillus cereus, Bacillus subtilis, Bacillus thuringiensis, and Bacillus stearothermophilus, Bacteroides sp. (for example, Bacteroides fragilis, Bartonella sp. (for example, Bartonella bacilliformis and Bartonella henselae, Bifidobacterium sp., Bordetella sp. (for example Bordetella pertussis, Bordetella parapertussis and Bordetella bronchiseptica, Borrelia sp. (for example, Borrelia recurrentis and Borrelia burgdorferi, Brucella sp. (for example, Brucella abortus, Brucella canis, Brucella melintensis and Brucella suis, Burkholderia sp. (for example, Burkholderia pseudomallei, and Burkholderia cepacia, Campylobacter sp. (for example, Campylobacter jejuni, Campylobacter coli, Campylobacter lar, and Campylobacter fetus, Capnocytophaga sp., Cardiobacterium hominis, Chlamydia trachomatis, Chlamydophila pneumonia, Chlamydophila psittaci, Citrobacter sp. Coxiella burnetii, Corynebacterium sp. (for example, Corynebacterium diphtheria), Corynebacterium jeikeum, and Corynebacterium, Clostridium sp. (for example, Clostridium perfringens, Clostridium difficile, Clostridium botulinum, and Clostridium tetani, Eikenella corrodens, Enterobacter sp. (for example, Enterobacter aerogenes, Enterobacter agglomerans, Enterobacter cloacae, and enterotoxigenic E. coli, enteroinvasive E. coli, enteropathogenic E. coli, enterohemorrhagic E. coli, enteroaggregative E. coli, and Escherichia coli including opportunistic E. coli such as uropathogenic E. coli, Enterococcus sp. (for example Enterococcus faecalis and Enterococcus faecium), Ehrlichia sp. (for example, Ehrlichia chafeensia and Ehrlichia canis, Erysipelothrix rhusiopathiae, Eubacterium sp., Francisella tularensis, Fusobacterium nucleatum, Gardnerella vaginalis, Gemella morbillorum, Haemophilus sp. (for example, Haemophilus influenzae, Haemophilus ducreyi, Haemophilus aegyptius, Haemophilus parainfluenzae, Haemophilus haemolyticus, and Haemophilus parahaemolyticus, Helicobacter sp. for example, Helicobacter pylori, Helicobacter cinaedi, and Helicobacter fennelliae, Kingella kingii, Klebsiella sp. (for example, Klebsiella pneumoniae, Klebsiella granulomatis, and Klebsiella oxytoca, Lactobacillus sp., Listeria monocytogenes, Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. for example, Mycobacterium leprae, Mycobacterium intracellulare, Mycobacterium avium, Mycobacterium bovis, and Mycobacterium marinum, Mycoplasm sp. (for example, Mycoplasma pneumoniae, Mycoplasma hominis, and Mycoplasma genitalium, Nocardia sp. (for example, Nocardia asteroides, Nocardia cyriacigeorgica, and Nocardia brasiliensis, Neisseria sp. (for example, Neisseria gonorrhoeae, and Neisseria meningitidis, Pasteurella multocida, Plesiomonas shigelloides, Prevotella sp., Porphyromonas sp., Prevotella melaninogenica, Proteus sp. (for example, Proteus vulgaris and Proteus mirabilis, Providencia sp. (for example, Providencia alcalifaciens, Providencia rettgeri, and Providencia stuartii, Pseudomonas aeruginosa, Propionibacterium acnes, Rhodococcus equi, Salmonella sp. (for example, Salmonella enterica, Salmonella typhi, Salmonella paratyphi, Salmonella enteritidis, Salmonella cholerasuis, and Salmonella typhimurium, Serratia sp. (for example, Serratia marcesans, and Serratia liquifaciens, Shigella sp. (for example, Shigella dysenteriae, Shigella flexneri, Shigella boydii and Shigella sonnei, Staphylococcus sp. (for example, Staphylococcus aureus, Staphylococcus epidermidis, Staphylococcus hemolyticus, Staphylococcus saprophyticus, Streptococcus sp. (for example, Streptococcus pneumoniae, Spectinomycin-resistant serotype 6B Streptococcus pneumoniae, Streptomycin-resistant serotype 9V Streptococcus pneumoniae, Erythromycin-resistant serotype 14 Streptococcus pneumoniae, Optochin-resistant serotype 14 Streptococcus pneumoniae, Rifampicin-resistant serotype 18C Streptococcus pneumoniae, Tetracycline-resistant serotype 19F Streptococcus pneumoniae, Penicillin-resistant serotype 19F Streptococcus pneumoniae and Trimethoprim-resistant serotype 23F Streptococcus pneumoniae, Chloramphenicol-resistant serotype 4 Streptococcus pneumoniae, Streptomycin-resistant serotype 9V Streptococcus pneumoniae, Optochin-resistant serotype 14 Streptococcus pneumoniae, Rifampicin-resistant serotype 18C Streptococcus pneumoniae, Penicillin-resistant serotype 19F Streptococcus pneumoniae) or Trimethoprim-resistant serotype 23F Streptococcus pneumoniae, Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equisimilis, Group D streptococci, Streptococcus bovis, Group F streptococci and Streptococcus anginosus, Group G streptococci, Spirillum minus, Streptobacillus moniliformi, Treponema sp. (for example, Treponema carateum, Treponema petenue, Treponema pallidum, and Treponema endemicum, Tropheryma whippelii, Ureaplasma urealyticum, Veillonella sp., Vibrio sp. (for example, Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii, Yersinia sp. (for example, Yersinia enterocolitica and Yersinia pestis and Xanthomonas maltophilia).

In one embodiment of the present invention, the antibacterial composition is an antibacterial pharmaceutical composition.

The pharmaceutical composition of the present invention may further contain a pharmaceutically acceptable carrier in addition to the composition as an active ingredient.

The pharmaceutically acceptable additive contained in the pharmaceutical composition of the present invention may be commonly used in the formulation and includes, but is not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate and mineral oil.

In addition to the ingredients, the pharmaceutical composition of the present invention may further contain lubricants, wetting agents, sweeteners, flavoring agents, emulsifiers, suspending agents, preservatives, or the like. Suitable pharmaceutically acceptable carriers and agents are described in detail in Remington's Pharmaceutical Sciences (19th ed., 1995).

The pharmaceutical composition of the present invention may be administered orally or parenterally, for example, intrathecally, intravenously, subcutaneously, intradermally, intramuscularly, intraperitoneally, intrasternally, intratumorally, intranasally, intracerebrally, intracranially, intrapulmonarily, and intrarectally, but is not limited thereto.

That is, the pharmaceutically effective amount of the pharmaceutical composition of the present invention may vary depending on the formulation method, administration method, age, weight, gender, pathological condition, diet, administration time, administration route, excretion rate and reaction sensitivity of the patient. Ordinary skilled physicians can easily determine and prescribe an effective dosage (pharmaceutically effective amount) for desired treatment or prevention. According to a preferred embodiment of the present invention, the daily dose of the pharmaceutical composition of the present invention is about 0.0001 to about 100 mg/kg.

As used herein, the term “pharmaceutically effective amount” means an amount sufficient to prevent or treat the disease.

As used herein, the term “prevention” refers to any action that suppresses or delays the onset of a disease by administration of the pharmaceutical composition according to the present invention. As used herein, the term “treatment” refers to any action that reduces, inhibits, ameliorates or removes disease conditions.

The pharmaceutical composition of the present invention is formulated into a unit dose form or packaged into a multiple dose container using a pharmaceutically acceptable carrier and/or excipient in accordance with a method that can be easily performed by those skilled in the art. The formulation may be prepared in a variety of forms, such as an oral drug or injection, may be in the form of a solution, suspension or emulsion in an oil or aqueous medium, or in the form of an extract, a powder, a suppository, a powder, a granule, a tablet or a capsule, and may further contain a dispersant or stabilizer.

In another aspect, the present invention is directed to a method for preventing or treating bacterial infection, including administering to a subject an antibacterial composition containing the protein complex of the present invention described above.

As used herein, the term “bacterial infection” refers to a disease caused by bacterial infection. Examples of the bacterial infection include tuberculosis, pneumonia, food poisoning, sepsis, toxic shock syndrome, scarlet fever, diphtheria, brucellosis, listeriosis, typhoid fever, paratyphoid fever, cholera, botulism, tetanus, leprosy, leptospirosis, and the like, caused by the pathogens describe above, but are not limited thereto.

As used herein, the term “administration” or “administering” means directly administering a therapeutically or prophylactically effective amount of the composition of the present invention to a subject suffering from, or likely to suffer from, the disease of interest, so that the same amount accumulates in the body of the subject.

As used herein, the term “therapeutically effective amount” refers to the content of the composition sufficient to impart a therapeutic or prophylactic effect to the subject to whom the composition is administered, and is meant to include a “prophylactically effective amount.”

In addition, as used herein, the term “subject” refers to mammals including humans, mice, rats, guinea pigs, dogs, cats, horses, cows, pigs, monkeys, chimpanzees, baboons, and rhesus monkeys. Most specifically, the subject of the present invention is a human.

The method for preventing or treating bacterial infection of the present invention includes administering a pharmaceutical composition, which is an aspect of the present invention, and thus duplicate description is omitted to avoid excessive redundancy in the specification.

In another aspect, the present invention is directed to a feed additive containing the protein complex described above.

The feed additive may be in a liquid or dry form, for example, in a dried powder.

In addition, the feed additive may further contain ordinary additives that can increase the preservability of the feed in addition to the antibiotic.

Here, the feed additives include, but are not limited to, commercially available feed, grains, roots, fruits, food processing by-products, algae, fibers, pharmaceutical by-products, oils and fats, starches, cucurbits, grain by-products, proteins, inorganics, oils and fats, minerals, single-cell proteins, zooplankton, leftover food, and the like.

In another aspect, the present invention is directed to a food additive or drinking water additive containing the protein complex as an active ingredient. By mixing and supplying the protein complex with drinking water, the number of strains in drinking water can be reduced.

The protein complex of the present invention functions to deliver endolysin into the target strain using the botulinum toxin translocation domain and thus has lytic activity not only against Gram-positive bacteria but also against Gram-negative bacteria, which endolysin has difficulty acting upon, based on this function. Further, the usability of the protein delivery system can be improved using a receptor-binding protein.

The antibacterial activity of the protein complex of the present invention is based on the protein delivery system of the botulinum toxin translocation domain, and the endolysin and receptor-binding protein may be selected in accordance with the target strain, and exhibits antibacterial activity, regardless of the type of endolysin and receptor-binding protein.

Furthermore, when producing a protein complex by reacting (protein trans-splicing) a protein fragment containing a botulinum toxin translocation domain with a protein fragment containing endolysin, than when producing a full-length protein complex at once, the protein complex is not easily denatured during the preparation process and is not vulnerable to chemical stress, and the preparation efficiency thereof is improved.

In another aspect, the present invention is directed to a method for screening an antibacterial protein complex including reacting the protein complex with a target strain, and determining whether or not the protein complex exhibits antibacterial activity against the target strain.

Through the screening method, by reacting the protein complex containing receptor-binding protein candidates and endolysin candidates with the target strain, and determining whether or not the candidates exhibit antibacterial activity against the target strain can be determined, whether or not the candidates can be used as receptor-binding proteins and endolysin can be determined.

The method for determining whether or not the candidate exhibits the antibacterial activity is not particularly limited and a known method may be used singly or in combination. According to an embodiment of the present invention, the method may be performed through a spotting assay.

Hereinafter, the present invention will be described in more detail with reference to the following examples. It will be obvious to those skilled in the art that these examples are provided only for better understanding of the present invention and thus should not be construed as limiting the scope of the present invention based on the subject matter of the present invention.

Throughout this specification, “percentage (%)” used to indicate a concentration of a specific substance means (weight/weight) % for solid/solid, (weight/volume) % for solid/liquid, and (volume/volume) % for liquid/liquid, unless mentioned otherwise.

<Botulinum Toxin Translocation Domain, Endolysin, Receptor-Binding Protein, Intein, SpyTag/Spycatcher for Production of Translysin (Protein Complex)>

Botulinum toxin translocation domain (Table 1), endolysin (Table 2) for preparation of translysin, receptor-binding protein (Table 3), intein (Table 4), and SpyTag/Spycatcher (Table 5) are listed as follows.

TABLE 1
Botulinum toxin translocation domain (H_{N})

| | | | | NCBI |
|---|---|---|---|---|
| | Predicted | Size | | Accession |
| Name | function | (a.a) | origin | # |
| BoNT A2 | Translocation | 422 | Clostridium | Q45894 |
| Translocation | domain | | botulinum | (449-870 |
| domain | | | strain | region) |
| | | | Kyoto/Type A2 | |
| BoNT C1 | Translocation | 416 | Clostridium | P18640 |
| Translocation | domain | | botulinum C | (450-865 |
| domain | | | bacteriophage | region) |
| BoNT E1 | Translocation | 397 | Clostridium | Q00496 |
| Translocation | domain | | botulinum | (423-819 |
| domain | | | | region) |

TABLE 2
Endolysin for production of translysin

| | Predicted | Size | | NCBI |
|---|---|---|---|---|
| Name | functions | (a.a) | Origin | Accession # |
| lys | L-alanyl-D- | 137 | Escherichia | YP_006868 |
| | glutamate | | virus T5 | |
| | endopeptidase | | | |
| | (M15) | | | |
| LysPA26 | Muramidase | 145 | Pseudomonas | KY615005 |
| | (GH24) | | phage JD010 | |
| PB1_gp48 | Chitinase | 220 | Pseudomonas | NC_011810 |
| | (GH19) | | phage PB1 | |
| LysAB2_P3 | Chitinase | 185 | Acinetobacter | HM755898 |
| | (GH19) | | phage phiAB2 | |
| PlyF307 | Muramidase | 146 | Acinetobacter | KJ740396 |
| | (GH24) | | phage RL-2015 | |
| AcLys | Muramidase | 184 | Acinetobacter | WP_000208716 |
| | (GH24) | | baumannii AB5075 | |
| | | | prophage | |
| PlyPA03 | Muramidase | 144 | Pseudomonas | WP_070344501 |
| | (GH24) | | aeruginosa | |
| | | | prophage | |
| PlyPA91 | Muramidase | 154 | Pseudomonas | CRR10611 |
| | (GH24) | | aeruginosa | |
| | | | prophage | |
| Abtn-4 | Chitinase | 185 | Acinetobacter | AVP40474 |
| | (GH19) | | phage | |
| | | | VB_AbaP_D2 | |
| WCHABP1_gp01 | N-acetylmuramidase | 171 | Acinetobacter | AST13128 |
| | (GH108) | | phage WCHABP1 | |
| WCHABP12_gp19 | Chitinase | 202 | Acinetobacter | ARB06760 |
| | (GH19) | | phage WCHABP12 | |
| gh-1p12 | N- | 146 | Pseudomonas | NP_813758 |
| | acetylmuramoyl- | | phage gh-1 | |
| | L- | | | |
| | alanine amidase | | | |
| B3ORF25 | Membrane-bound | 264 | Pseudomonas | YP_164061 |
| | lytic murein | | phage B3 | |
| | transglycosylase | | | |
| | F | | | |
| phi-13Sp4 | P5 muramidase | 245 | Pseudomonas | NP_690810 |
| | | | phage phi13 | |
| phi-6S_4 | Peptidase_U40 | 220 | Pseudomonas | NP_620343 |
| | | | phage phi6 | |
| KP27_166 | L-alanyl-D- | 131 | Klebsiella | AEX26632 |
| | glutamate | | phage | |
| | endopeptidase | | VB_KpnM_KP27 | |
| KP13_gp066 | Muramidase | 160 | Klebsiella | AZF89867 |
| | (GH24) | | phage | |
| | | | VB_KpnS_Kp13 | |
| BI057_gp221 | T4-like | 164 | Shigella phage | YP_009279119 |
| | lysozyme | | SHFML-26 | |
| LPSE_00024 | Soluble Lytic | 162 | Salmonella | APU02985 |
| | Transglycosylases | | phage LPSE1 | |
| STP4a_120 | Soluble Lytic | 166 | Salmonella | AHJ86974 |
| | Transglycosylases | | phage STP4-a | |
| Lys68 | Soluble Lytic | 162 | Salmonella | AHY18890 |
| | Transglycosylases | | phage phi68 | |
| SPN1S_0028 | Chitinase | 209 | Salmonella | YP_005098003 |
| | (GH19) | | phage SPN1S | |
| P22gp66 | Lysozyme | 146 | Salmonella | NP_059622 |
| | | | virus P22 | |

TABLE 3
Receptor-binding protein (RBP)

| | Predicted | Size | | NCBI |
|---|---|---|---|---|
| Name | functions | (a.a) | origin | Accession # |
| PRD1_04 | Adsorption | 591 | Enterobacteria | NC_001421 |
| | protein | | phage PRD1 | |
| P1301_0153 | Receptor- | 593 | Bacteriophage | ASU02516 |
| | binding | | T5-like | |
| | protein | | chee130_1 | |
| P24_0149 | Receptor- | 585 | Bacteriophage | NC_047885 |
| | binding | | T5-like chee24 | |
| | protein | | | |
| Pb5 | Receptor- | 640 | Escherichia | NC_005859 |
| | binding | | virus T5 | |
| | tail protein | | | |
| AbTJ_gp52 | Tail fiber | 202 | Acinetobacter | QAU04145 |
| | protein | | phage AbTJ | |
| AbTJ_gp53 | Tail fiber | 699 | Acinetobacter | QAU04146 |
| | protein | | phage AbTJ | |
| phiAB6_gp40 | Tail fiber | 699 | Acinetobacter | ALA12264 |
| | | | phage phiAB6 | |
| S | Tail fiber | 504 | Escherichia | NC_000929 |
| | protein | | phage Mu | |
| A318_gp060 | Receptor- | 585 | Escherichia phage | NC_017969 |
| | binding | | vB_EcoS_AKFV33 | |
| | tail protein | | | |
| rv5_gp030 | Tail fiber | 347 | Escherichia | YP_002003532 |
| | protein | | phage rV5 | |
| rv5_gp033 | Putative | 346 | Escherichia | YP_002003535 |
| | tail fiber | | phage rV5 | |
| | protein | | | |
| PaoP5_075 | Structural | 243 | Pseudomonas | YP_009224766 |
| | protein | | phage PaoP5 | |
| BH773_gp153 | Putative | 670 | Pseudomonas | YP_009273830 |
| | tail fiber | | phage K5 | |
| | protein | | | |
| AU075_gp145 | Putative | 499 | Pseudomonas | YP_009186965 |
| | tail fiber | | phage C11 | |
| | protein | | | |
| CPT_Sugarland_191 | Receptor- | 658 | Klebsiella phage | ATW62004 |
| | binding | | Sugarland | |
| | protein | | | |
| JIPhKp127_0170 | Receptor- | 658 | Klebsiella phage | QFR57578 |
| | binding | | JIPh_Kp127 | |
| | protein | | | |
| AmPhEK80_0178 | Receptor- | 658 | Klebsiella phage | QFR57428 |
| | binding | | AmPh_EK80 | |
| | protein | | | |
| P22_gp19 | Tail spike | 667 | Salmonella phage | NC_002371 |
| | protein | | P22 | |
| DET7_207 | Tail spike | 708 | Salmonella phage | YP_009140379 |
| | protein | | Det7 | |
| HWD08_gp154 | Receptor- | 585 | Salmonella phage | YP_009856550 |
| | binding | | L6jm | |
| | tail protein | | | |
| BI021_gp088 | Receptor- | 595 | Salmonella phage | YP_009283429 |
| | binding | | NR01 | |
| | protein | | | |
| HWD21_gp023 | Receptor- | 640 | Salmonella phage | YP_009857757 |
| | binding | | oldekolle | |
| | tail protein | | | |
| HWC41_gp146 | Receptor- | 593 | Salmonella phage | YP_009848613 |
| | binding | | SE24 | |
| | protein | | | |
| HOS12_gp017 | Receptor- | 585 | Salmonella phage | YP_009792475 |
| | binding | | SP01 | |
| | protein | | | |
| HOU44_gp075 | Receptor- | 593 | Salmonella phage | YP_009815133 |
| | binding | | STG2 | |
| | protein | | | |
| I133_gp019 | Receptor | 249 | Salmonella phage | YP_007501289 |
| | recognition | | vB_SenM-S16 | |
| | protein | | | |
| HWC50_gp066 | Receptor- | 585 | Salmonella virus | YP_009849685 |
| | binding | | VSe12 | |
| | tail protein | | | |
| HOS34_gp106 | Receptor- | 585 | Shigella phage | YP_009794581 |
| | binding | | SSP1 | |
| | tail protein | | | |

TABLE 4
Intein

| | | | SEQ | |
|---|---|---|---|---|
| | | | ID | |
| Name | Domain | Sequence | NO: | Remarks |
| Gp41.1 | Int-N | CLDLKTQVQTPQGMKEISNIQVGDLVLSNTGYNEVL | 1 | |
| | | NVFPKSKKKSYKITLEDGKEIICSEEHLFPTQTGEM | | |
| | | NISGGLKEGMCLYVKE | | |
| | Int-C | MMLKKILKIEELDERELIDIEVSGNHLFYANDILTH | 2 | |
| | | N | | |
| Cfa | Int-N | CLSYDTEILTVEYGFLPIGKIVEERIECTVYTVDKN | 3 | |
| | | GFVYTQPIAQWHNRGEQEVFEYCLEDGSIIRATKDH | | |
| | | KFMTTDGQMLPIDEIFERGLDLKQVDGLP | | |
| | Int-C | MVKIISRKSLGTQNVYDIGVEKDHNFLLKNGLVASN | 4 | |
| NRDJ-1 | Int-N | CLVGSSEIITRNYGKTTIKEVVEIFDNDKNIQVLAF | 5 | |
| | | NTHTDNIEWAPIKAAQLTRPNAELVELEIDTLHGVK | | |
| | | TIRCTPDHPVYTKNRGYVRADELTDDDELVVA | | |
| | Int-C | MIEAKTYIGKLKSRKIVSNEDTYDIQTSTHNFFAND | 6 | |
| | | ILVHN | | |
| IMPDH-1 | Int-N | CFVPGTLVNTENGLKKIEEIKVGDKVFSHTGKLQEV | 7 | |
| | | VDTLIFDRDEEIISINGIDCTKNHEFYVIDKENANR | | |
| | | VNEDIHLFARWVVHAEELDMKKHLLIELE | | |
| | Int-C | MKFKLKEITSIETKHYKGKVHDLTVNQDHSYNVRGT | 8 | |
| | | VVHN | | |
| Npu | Int-N | CLSYETEILTVEYGLLPIGKIVEKRIECTVYSVDNN | 9 | DnaE |
| | | GNIYTQPVAQWHDRGEQEVFEYCLEDGSLIRATKDH | | |
| | | KMTVDGQMLPIDEIFERELDLMRDNLPN | | |
| | Int-C | MIKIATRKYLGKQNVYDIGVERDHNFALKNGFIASN | 10 | |
| Ssp | Int-N | CLSFGTEILTVEYGPLPIGKIVSEEINCSVYSVDPE | 11 | DnaE |
| | | GRVYTQAIAQWHDRGEQEVLEYELEDGSVIRATSDH | | |
| | | RFLTTDYQLLAIEEIFARQLDLLTLENIKQTEEALD | | |
| | | NHRLPFPLLDAGTIK | | |
| | Int-C | MVKVIGRRSLGVQRIFDIGLPQDHNFLLANGAIAAN | 12 | |
| Rma | Int-N | CLAGDTLITLADGRRVPIRELVSQQNFSVWALNPQT | 13 | DnaB |
| | | YRLARVSRARVSRAFCTGIKPVYRLTTRLGRSIRAT | | |
| | | ANHRFLTQGWKRVDELQPGDYLALPRRIPTASTPTL | | |
| | | TEAELALLGHLIGD | | |
| | Int-C | MWDPIVSIEPDGVEEVFDLTVPGPHNFVADNIIAGN | 14 | |
| | | S | | |
| Ppu | Int-N | CISKFSHIMWSHVSKPLFNFSIKKSHMHNGNKNIYQ | 15 | DnaB |
| | | LLDQGEAFISRQDKKTTYKIRTNSEKYLELTSNHKI | | |
| | | LTLRGWQRCDQLLCNDMITTQIGFELSRKKKYLLNC | | |
| | | IPFSLCNFET | | |
| | Int-C | MLANINISNFQNVEDFAANPIPNFIANNIIVHNS | 16 | |
| Gp41.8 | Int-N | CLSLDTMVVINGKAIEIRDVKVGDWLESECGPVQVT | 17 | |
| | | EVLPIIKQPVFEIVLKSGKKIRVSANHKFPTKDGLK | | |
| | | TINSGLKVGDFLRSRA | | |
| | Int-C | MCEIFENEIDWDEIASIEYVGVEETIDINVINDRLF | 18 | |
| | | FANGILTHN | | |
| NrdA-2 | Int-N | CLTGDAKIDVLIDNIPISQISLEEVVNLFNEGKEYV | 19 | |
| | | LSYNIDTKEVEYKEISDAGLISESAEVLEIIDEETG | | |
| | | QKIVCTPDHKVYTLNRGYVSAKDLKEDDELVES | | |
| | Int-C | MGLKIIKRESKEPVFDITKVKDNSNFFANNILVHN | 20 | |

TABLE 5
SpyTag & Spycatcher

| | | | SEQ ID | |
|---|---|---|---|---|
| Name | Domain | Sequence | NO: | Remarks |
| Spy001 | Catcher | GAMVDTLSGLSSEQGQSGDMTIEEDSATHIKF | 32 | |
| | | SKRDEDGKELAGATMELRDSSGKTISTWISDG | | |
| | | QVKDFYLYPGKYTFVETAAPDGYEVATAITFT | | |
| | | VNEQGQVTVNGKATKGDAHI | | |
| | Tag | AHIVMVDAYKPTK | 33 | |
| Spy002 | Catcher | AMVTTLSGLSGEQGPSGDMTTEEDSATHIKFS | 34 | |
| | | KRDEDGRELAGATMELRDSSGKTISTWISDGH | | |
| | | VKDFYLYPGKYTFVETAAPDGYEVATAITFTV | | |
| | | NEQGQVTVNGEATKGDAHTGSSGS | | |
| | Tag | VPTIVMVDAYKRYK | 35 | |
| Spy003 | Catcher | VTTLSGLSGEQGPSGDMTTEEDSATHIKFSKR | 36 | |
| | | DEDGRELAGATMELRDSSGKTISTWISDGHVK | | |
| | | DFYLYPGKYTFVETAAPDGYEVATPIEFTVNE | | |
| | | DGQVTVDGEATEGDAHT | | |
| | Tag | RGVPHIVMVDAYKRYK | 37 | |

However, the contents described in Tables 1 to 5 are provided for illustrating the present invention in more detail and Tables 1 to 5 should not be construed as limiting the scope of the present invention.

### Example 1: Verification of Intracellular Expression and Lytic Effect of Full-Length Translysin

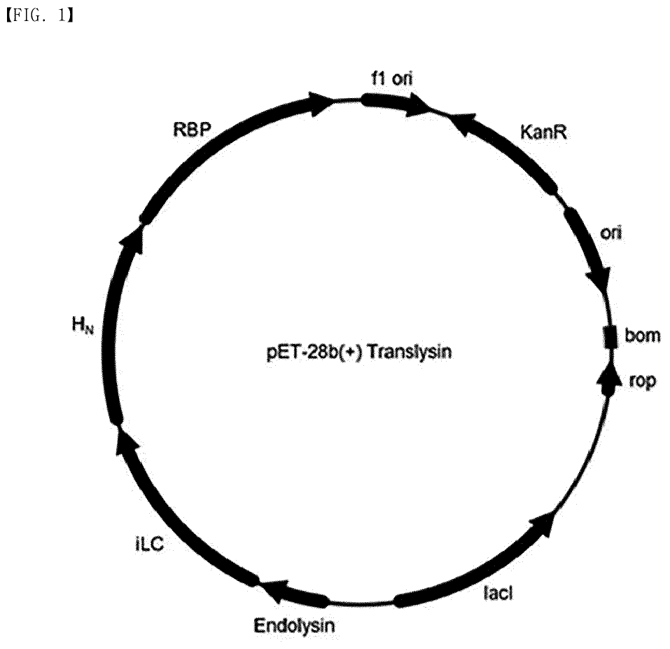

To express full-length translysin (endolysin-H_{N}-RBP) in the expression host, E. coli shuffle T7, a plasmid encoding the translysin was designed. The protein complex was designed to have endolysin (T5 lys) and RBP (Pb5) bound to the amino and carboxyl ends of the botulinum toxin H_{N} domain, respectively, and was inserted into a pET vector to produce a plasmid (FIG. 1). The sequence of the full-length translysin inserted into the vector is shown in SEQ ID NO: 21. The plasmid was inserted into an E. coli host at 42° C., cultured on LB (Luria-Bertani) agar medium supplemented with kanamycin at a concentration of 50 μg/mL and a transformed E. coli strain was selected.

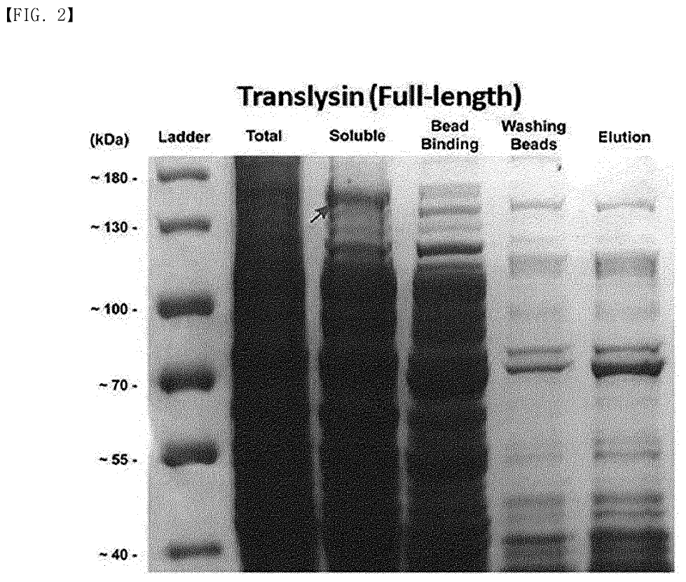

The selected transformed strain was inoculated into LB culture medium and sub-cultured. 1 mM IPTG (isopropyl β-D-1-thiogalactopyranoside) was added to the cultured solution, followed by incubation at 37° C. for 4 hours to induce expression of translysin, which was then identified by SDS gel electrolysis (FIG. 2). As control groups, a transformed strain of a plasmid encoding an endolysin fragment or innolysin (T5 lys-linker-Pb5) and a wild-type strain were used, and the expression of innolysin was identified by SDS gel electrophoresis (FIG. 3). The sequence of innolysin is shown in SEQ ID NO: 22. The activity of translysin was predicted by measuring the growth of the strain based on the turbidity of the culture medium after expression. It was predicted that, when the expressed protein complex passed through the cell membrane and lysed the peptidoglycan of the cell wall, the strain died and turbidity decreased.

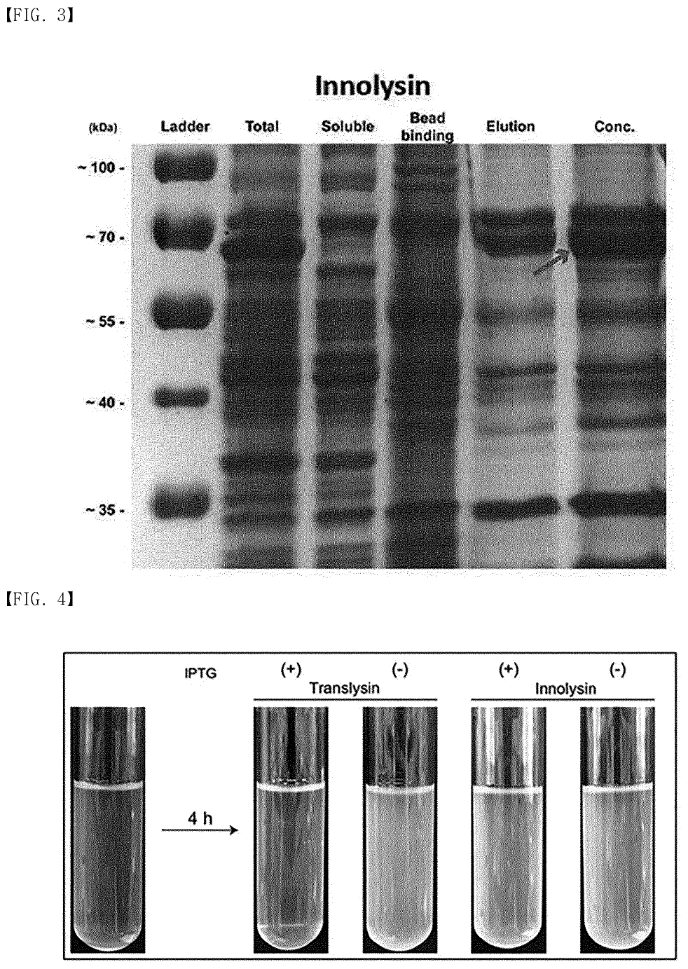

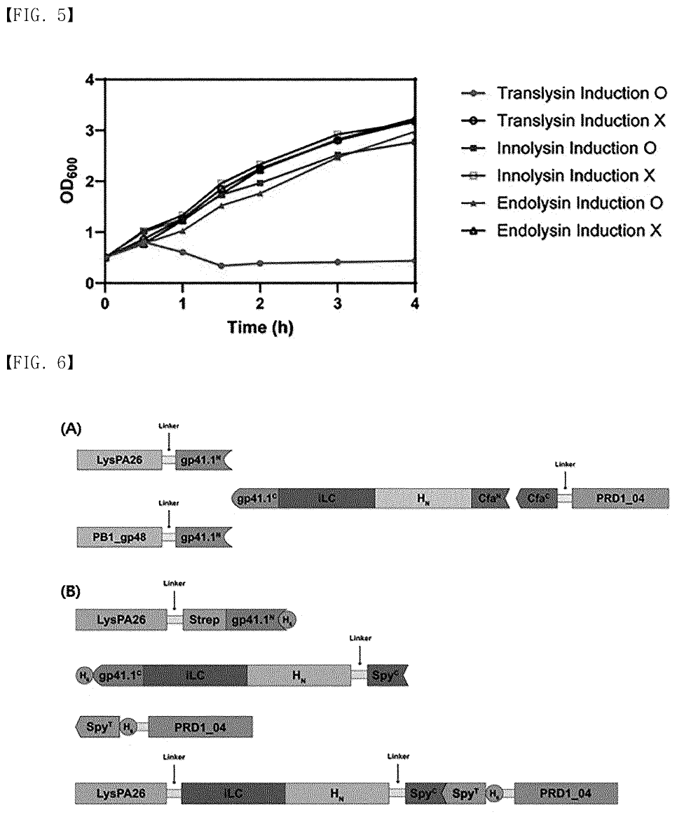

The result of expression showed that the strain expressing the endolysin fragment and innolysin did not exhibit a significant difference from the growth pattern of the wild-type strain, but when the translysin of the present invention was expressed, growth was significantly reduced, which indicates that membrane transfer occurred effectively (FIGS. 4 and 5).

### Example 2: Protein Trans-Splicing of Protein Fragment by Intein

During the expression process, the full-length translysin expressed in Example 1 has problems in that 1) it has difficulty in reaching a sufficient cell concentration because the expression host is lysed, 2) it is readily denatured and is thus expressed at a low amount in a soluble form because the folding of the expression product of about 180 kDa is not smooth, and 3) it is vulnerable to physical and chemical stress during separation and purification because the size of the expression product reaches about 180 kDa. Therefore, in order to overcome these problems, the present invention adopts a method of expressing and purifying endolysin, iLCH_{N} (inactivated botulinum toxin light chain+botulinum toxin translocation domain), and RBP as respective fragments linked to intein, and linking the fragments in vitro.

Gp41.1 intein and Cfa intein were selected from Table 4 in consideration of the linking conditions and reaction rates of inteins, and applied to endolysin-iLCH_{N} and iLCH_{N}-RBP binding, respectively. At this time, LysPA26 and PB1_gp48 in Table 2 were selected as endolysins, and PRD1_04 in Table 3 was selected as RBP.

The method for producing protein fragments is the same as in Example 1 and the sequences used to produce protein fragments are as follows:

LysPA26-Gp41.1N: SEQ ID NO: 23;
PB1_gp48-Gp41.1N: SEQ ID NO: 24;
Gp41.1C-iLCH _{N}-CfaN: SEQ ID NO: 25;
and
CfaC-PRD1_04: SEQ ID NO: 26.

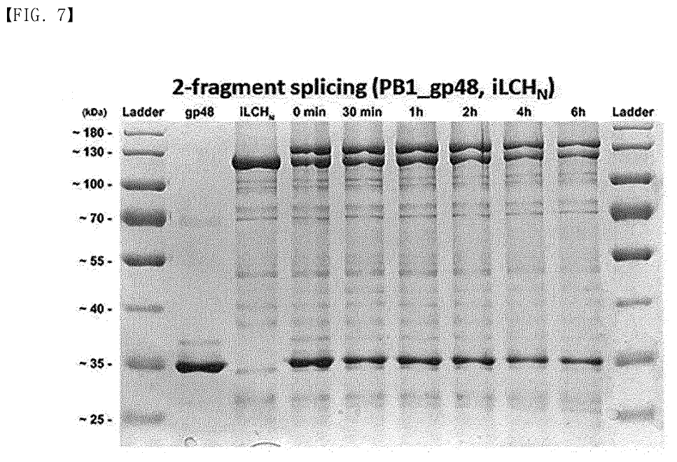

As a result, four protein fragments (LysPA26-Gp41.1N, 27.5 kDa; PB1_gp48-Gp41.1N, 35.3 kDa; Gp41.1C-iLCH_{N}-CfaN, 117.2 kDa; CfaC-PRD1_04, 69.3 kDa) were expressed and purified. A protein trans-splicing reaction was performed (FIG. 6(A)).

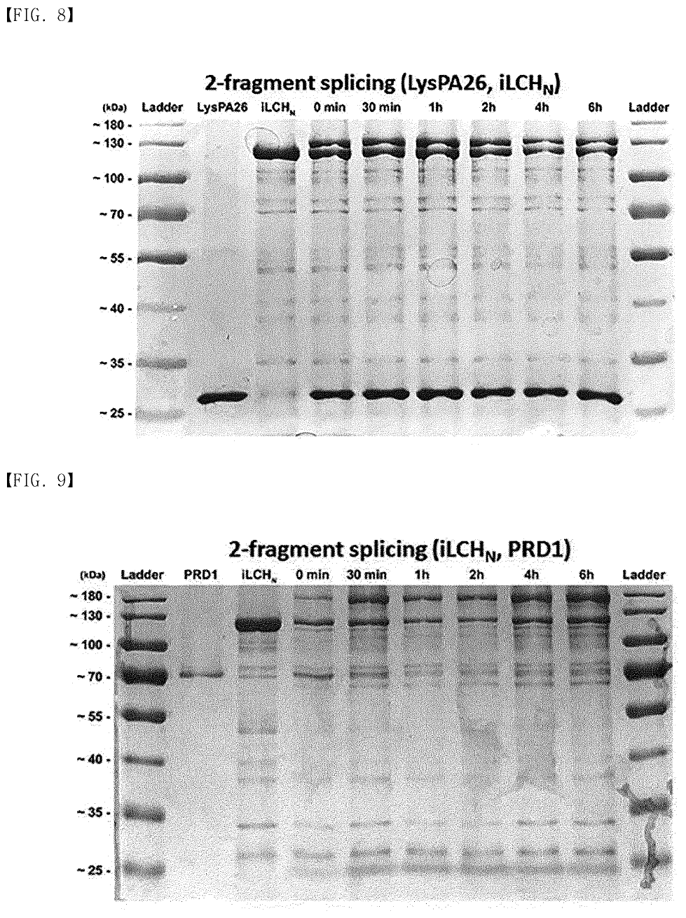

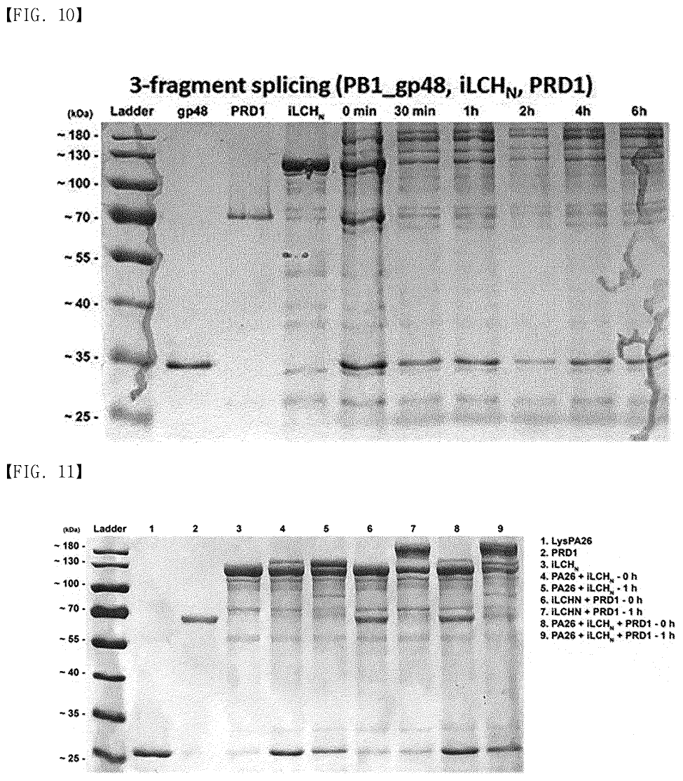

First, to determine the trans-splicing effects of Gp41.1 intein and Cfa intein, the fragments were mixed at a molar ratio of 1:1 and the reaction was performed. As a result, 128.7 kDa (LysPA26-iLCH_{N}-CfaN), 136.5 kDa (PB1_gp48-iLCH_{N}-CfaN), and 168.9 kDa (gp41.1C-LCH_{N}-PRD1_04) reaction products were obtained in buffer solution and saturated after 30 minutes (FIGS. 7 to 9). The result of the reaction performed by mixing the fragments at a molar ratio of 1:1:1 showed that the final product of 189.1 kDa, and the intermediate products of 136.5 kDa (PB1_gp48-iLCH_{N}-CfaN) and 168.9 kDa (Gp41.1C-iLCH_{N}-PRD1_04) were obtained in buffer solution regardless of the use of the reducing agent (FIG. 10).

### Example 3: Conversion of Protein Fragments to Full-Length Proteins by Intein and SpyTag/SpyCatcher

In the full-length translysin produced in Example 2, the intein can be removed, but there was a problem of low trans-splicing efficiency of about 25%. Therefore, in order to overcome this problem, the present invention adopted a method of expressing and purifying endolysin, iLCH_{N}, and RBP as respective fragments linked to intein and SpyTag/Spycatcher, and linking the fragments in vitro.

In consideration of the binding conditions and reaction rate of the intein and the mechanism of action of translysin, Gp41.1 intein was selected from Table 4 and applied to endolysin-iLCH_{N} binding. In consideration of the reaction speed of the SpyTag/Spycatcher, the 3^{rd} generation SpyTag/Spycatcher was selected from Table 5 and applied to iLCH_{N}-RBP binding. At this time, LysPA26 was selected as the endolysin from Table 2 and PRD1_04 was selected as the RBP from Table 3.

The method for producing protein fragments is the same as in Example 1 and the sequences used to produce protein fragments are as follows:

LysPA26-Gp41.1N: SEQ ID NO: 23;
Gp41.1C-iLCH_{N}-Spy ^{C}003: SEQ ID NO: 42;
and
Spy ^{T}003-PRD1_04: SEQ ID NO: 43

As a result, three protein fragments (LysPA26-Gp41.1N, 27.5 kDa; Gp41.1C-iLCH_{N}-Spy^{C}003, 117.8 kDa; Spy^{T}003-PRD1_04, 67.0 kDa) were expressed, purified and reacted into full-length translysin in vitro (FIG. 6(B)).

First, to determine the trans-splicing effect of Gp41.1 intein, the fragments were mixed at a molar ratio of 1:1 and the reaction was performed. The result showed that 129.1 kDa (LysPA26-iLCH_{N}-Spy^{C}003) was obtained in the buffer solution and saturated after 30 minutes (FIG. 11). In order to determine protein binding by SpyTag and Spycatcher, the fragments were mixed at a molar ratio of 1:1 and the reaction was performed. As a result, a reaction product of 184.8 kDa (gp41.1C-iLCH_{N}-Spy-PRD1_04) was obtained in the buffer solution (FIG. 11). The result of the reaction performed by mixing respective fragments at a molar ratio of 1:1:1 showed that a final product of 196.1 kDa and an intermediate product of 129.1 kDa (Gp41.1C-iLCH_{N}-Spy-PRD1_04) were produced in the buffer solution (FIG. 11).

### Example 4: Verification of Lytic Activity of Full-Length Translysin and Protein Fragments

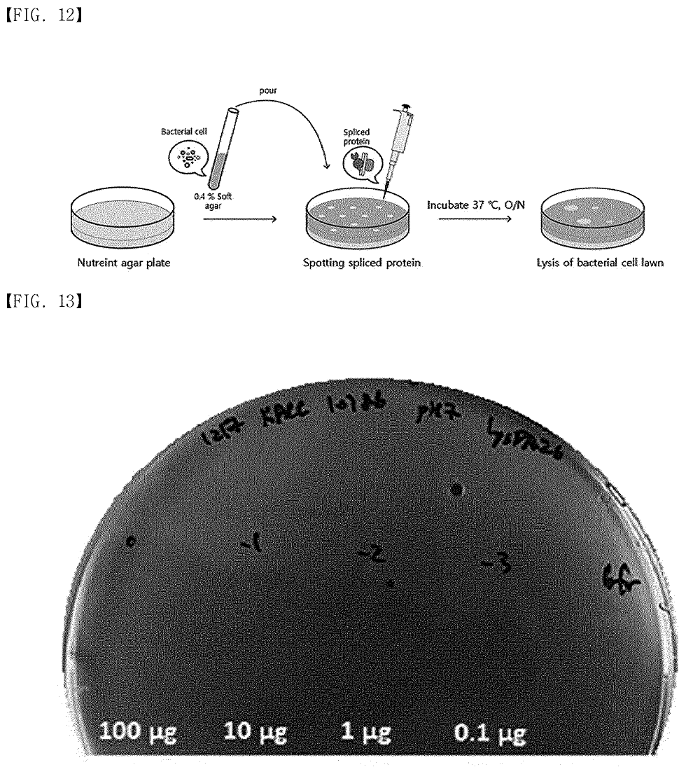

To determine the membrane transport and lytic activity of the full-length translysin produced in Examples 2 and 3, a spotting assay was performed on the target strain. The spotting assay is a method including overlaying a homogeneous mixture of 0.4% soft agar and the target strain on LB agar medium, drying the result, spotting the protein sample, culturing the sample, and determining death or growth inhibition patterns of strains on the lawn (FIG. 12).

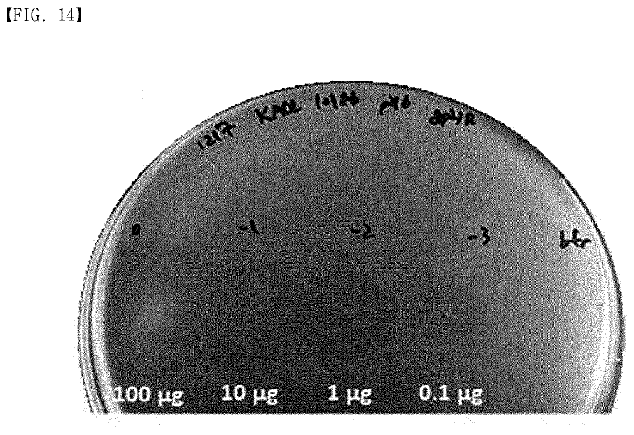

Spotting assays were performed in two groups. First, to verify the lytic activity of endolysin, an experiment was conducted using dead cells in which the outer membrane of the Pseudomonas aeruginosa KACC 10186 strain was destroyed. For this purpose, the target strain was cultured until the exponential phase (OD₆₀₀<2.0), sterilized at 121° C. using an autoclave, and then washed once with a buffer solution (50 mM Tris, 100 mM NaCl; pH 7, LysPA26/pH 6, PB1_gp48) to prepare a final 100-fold concentrated dead cell solution, diluted such that the turbidity of 0.4% soft agar was adjusted to OD₆₀₀ of 10, and 5 mL of the dilution was placed on the LB agar medium in a 90Ø Petri dish. After the soft agar was dried, 10 μL of each endolysin sample stock solution (10 μg/μL) and the dilution (1 μg/μL, 0.1 μg/μL, 0.01 μg/μL) were spotted, and a buffer solution was used as a negative control. The result of the test showed that both LysPA26 and PB1_gp48 endolysin fragments formed a lysis zone in the lawn, which indicates that the fragments exhibit lytic activity against the peptidoglycan of the target strain (FIGS. 13 and 14).

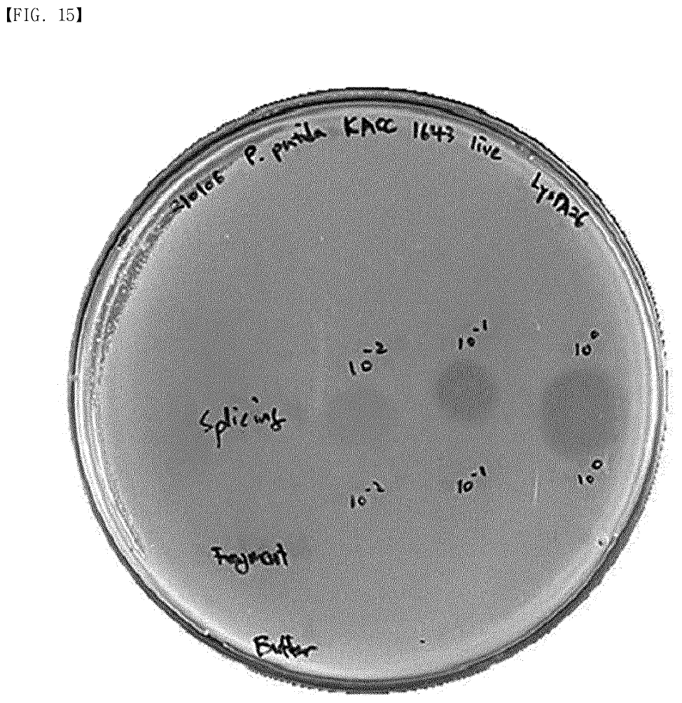

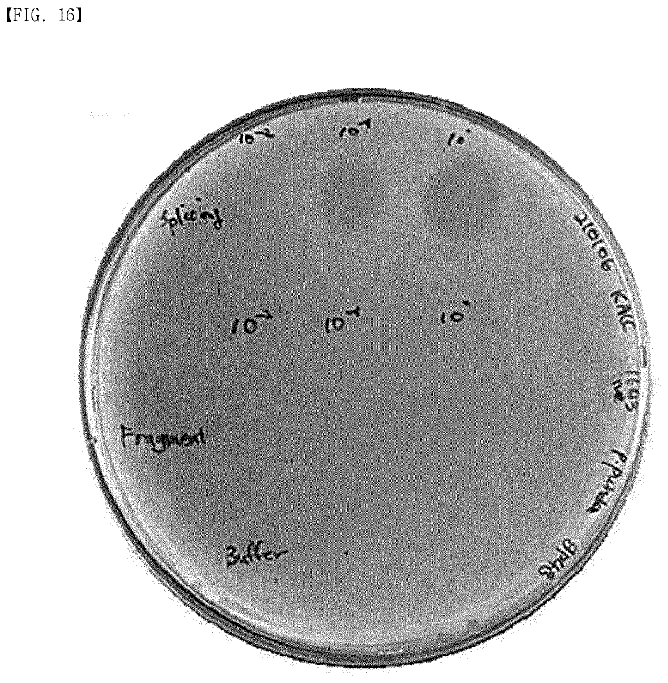

Then, to determine the membrane transport activity, an experiment was conducted by treating Pseudomonas putida KCTC 1643 viable cells with the protein sample under the same conditions. For this purpose, 100 μL of live bacteria cultured overnight at 37° C. in LB culture medium until a stationary phase were homogeneously mixed with 5 mL of 0.4% LB soft agar and overlayed to create an environment that promotes the growth of live bacteria. After the soft agar was dried, 10 μL of each of the stock solution (4 μg/μL) and diluted solutions (0.4 μg/μL, 0.04 μg/μL) of translysin (PB1_gp48-iLCH_{N}-PRD1_04, LysPA26-iLCH_{N}-PRD1_04) and endolysin fragments (PB1_gp48-Gp41.1N, LysPA26-Gp41.1N) were spotted, and a buffer solution was used as a negative control. The result of the experiment showed that no lytic action occurred when treated with each endolysin fragment, whereas a lysis zone was formed when treated with full-length translysin, which indicates that the protein was effectively delivered into the outer membrane of the target strain and lysed the cell. (FIGS. 15 and 16).

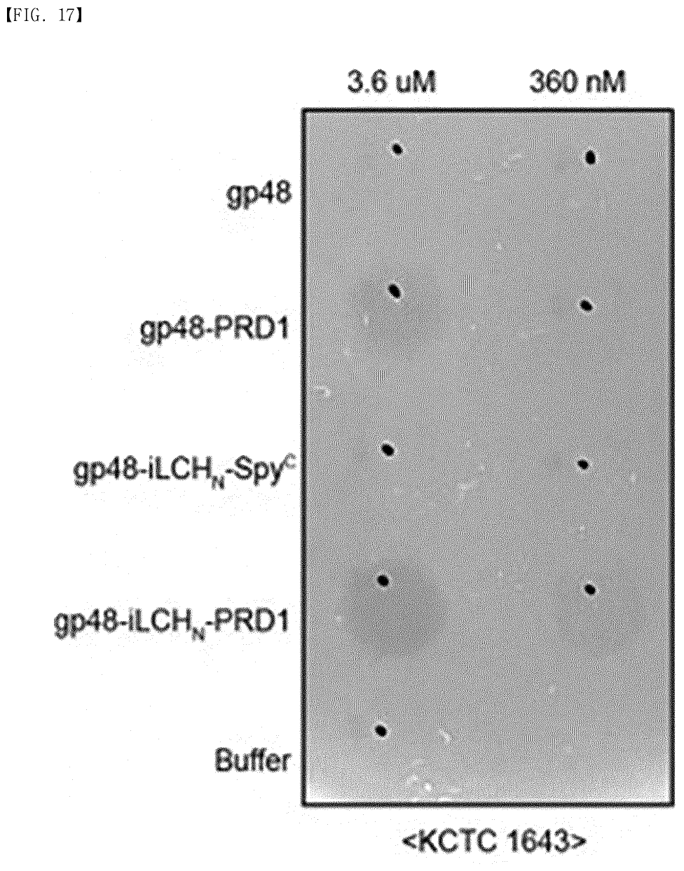

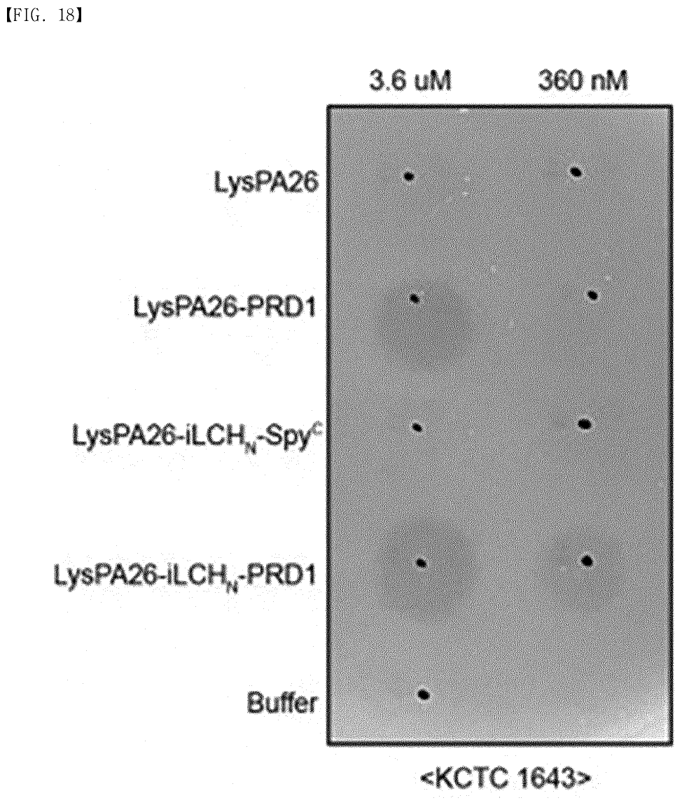

The result was treated with translysin (PB1_gp48-iLCH_{N}-PRD1_04, LysPA26-iLCH_{N}-PRD1_04) and endolysin fragments (PB1_gp48-Gp41.1N, LysPA26-Gp41.1N) assembled through SpyTag and Spycatcher, and innolysin (LysPA26-iLCH N-PRD1_04, PB1_gp48-iLCH N-PRD1_04) at 3.6 μM and 360 nM, respectively, under the same conditions. The result of the experiment showed that lytic activity did not occur when treated with each endolysin fragment, whereas when treated with full-length translysin and innolysin, a lytic zone was formed. The full-length translysin exhibited the effect at both 3.6 μM and 360 nM, whereas innolysin exhibited the effect at 3.6 μM (FIGS. 17 and 18).

### Example 5: Visualization of Lytic Activity of Translysin

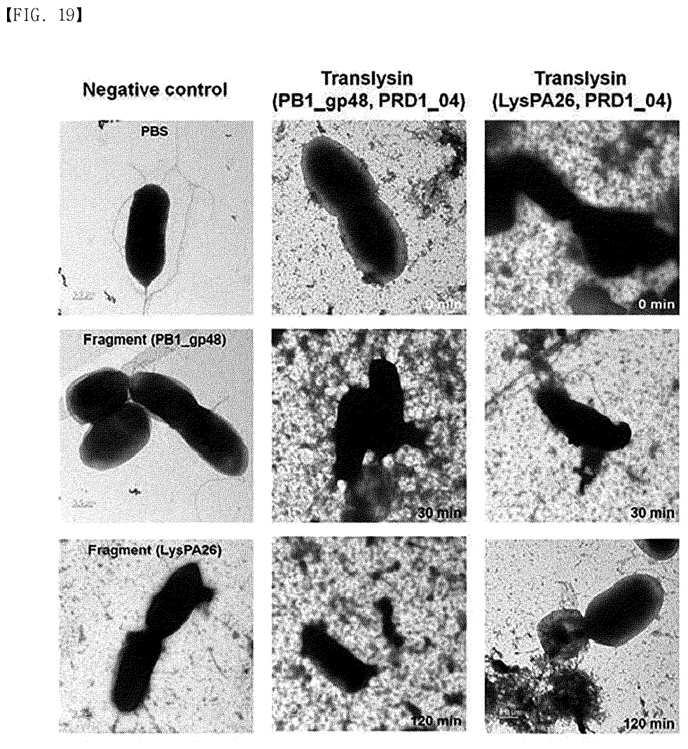

Transmission electron microscopy (TEM) was performed to visually detect the lytic activity of translysin verified in Example 4 (FIG. 19). For this purpose, the target strain P. putida KCTC 1643 was inoculated into the LB culture at a turbidity of OD₆₀₀=0.1, cultured at 37° C. for 2 hours until the early exponential phase (OD₆₀₀=1.0), and washed twice with a buffered saline solution to prepare live bacteria. Then, about 1 mL of the live bacteria solution was mixed with 370 nM translysin (PB1_gp48-iLCH_{N}-PRD1_04, LysPA26-iLCH_{N}-PRD1_04), cultured at 25° C., and 10 μL of the mixture was collected at a predetermined time point, fixed on a gold grid, and then stained with uranyl acetate for 10 seconds to prepare a TEM sample. A negative control sample was prepared in the same manner as above by treating a strain with each endolysin fragment (PB1_gp48-Gp41.1N, LysPA26-Gp41.1N) at a concentration of about 370 nM, or was prepared in the same manner as above using a strain without any treatment. As a result of analysis using an energy-filtering transmission electron microscope (Energy-Filtering TEM, 120 kv) (Carl Zeiss, Libra 120), no external damage was observed in the negative control, whereas the cell wall of the experimental group treated with translysin was damaged over time and the strain died (FIG. 19).

### Example 6: Quantification of Lytic Activity of Translysin

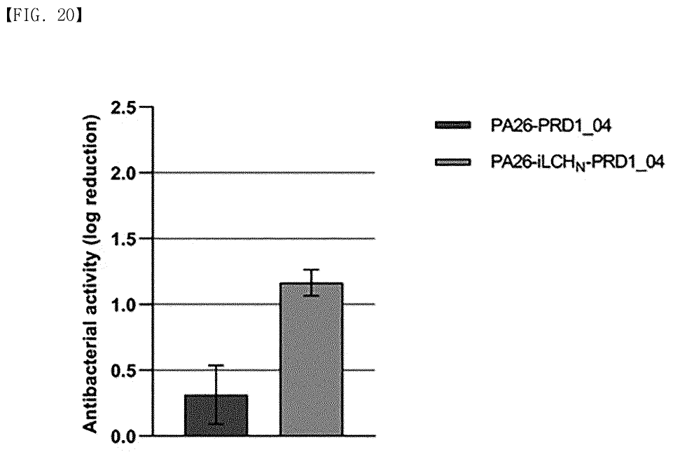

CFU (Colony Forming Unit) reduction assay was performed to numerically determine the lytic activity of translysin verified in Example 4. For this purpose, the target strain P. putida KCTC 1643 was inoculated into LB culture medium at a turbidity of OD₆₀₀=0.1, cultured at 37° C. for 1.5 hours until the early exponential phase (OD₆₀₀=0.6), washed three times with a 20 mM HEPES-NaOH (pH=7.4) solution, diluted 100 times and prepared as live bacteria. Then, 100 uL of translysin (LysPA26-iLCH_{N}-PRD1_04) or innolysin (LysPA26-PRD1) was mixed at a concentration of 30 μM with 100 μL of the live bacteria solution and reacted at 37° C. for 1 hour. As a negative control, a sample was prepared in the same manner as above using 20 mM Hepes-NaOH. Then, the result was diluted with an appropriate dilution factor, spread on LB agar medium, and cultured O/N at 37° C. Antibacterial activity was determined based on the difference in mean logarithmic cell concentration of translysin- or innolysin-treated samples compared to the negative control. The result of the experiment showed that translysin exhibited about twice the lytic activity of innolysin (FIG. 20).

### Example 7: Translysin Library-Based Antibacterial Substance Screening

A translysin screening method was designed to discover antibacterial substances against various strains using the protein delivery system of the present invention (FIG. 21). Specifically, the endolysins and RBPs in Tables 2 and 3 were combined to construct a translysin library, and viable cells of the target strain in Table 6 were homogeneously mixed with 0.4% LB soft agar to form a lawn. Then, novel antibacterial materials for each strain could be discovered by determining the lytic activity through spotting assay.

TABLE 6

| Candidate bacteria | Remarks |
|---|---|
| Pseudomonas aeruginosa PAO1 | |
| Pseudomonas aeruginosa KACC 10186 | |
| Pseudomonas aeruginosa ATCC 15692 | |
| Pseudomonas aeruginosa ATCC 27853 | |
| Pseudomonas putida KCTC 1643 | |
| Escherichia coli BL21 | |
| Escherichia coli MG1655 | |
| Escherichia coli ATCC 43889 | O157:H7, Stx2, |
| | hemolytic uremic |
| | syndrome |
| Escherichia coli ATCC 43890 | O157:H7, Stx1 |
| Escherichia coli ATCC 43895 | O157:H7, Stx1, Stx2, |
| | hemorrhagic colitis |
| Escherichia coli ATCC 35150 | O157:H7, hemorrhagic |
| | colitis |
| Escherichia coli ATCC 25922 | O6, clinical isolate |
| Salmonella Typhimurium SL1344 | |
| Salmonella Typhimurium UK-1 | |
| Salmonella Typhimurium 14028s | |
| Salmonella Typhimurium LT2 | |
| Salmonella Typhimurium DT104 | zoonotic pathogen, |
| | MDR |
| Klebsiella pneumoniae subsp. | |
| pneumoniae ATCC 10031 | |
| Klebsiella pneumoniae KCTC 2242 | |
| Klebsiella pneumoniae ATCC 13883 | |
| Klebsiella pneumoniae Revco 41 | |

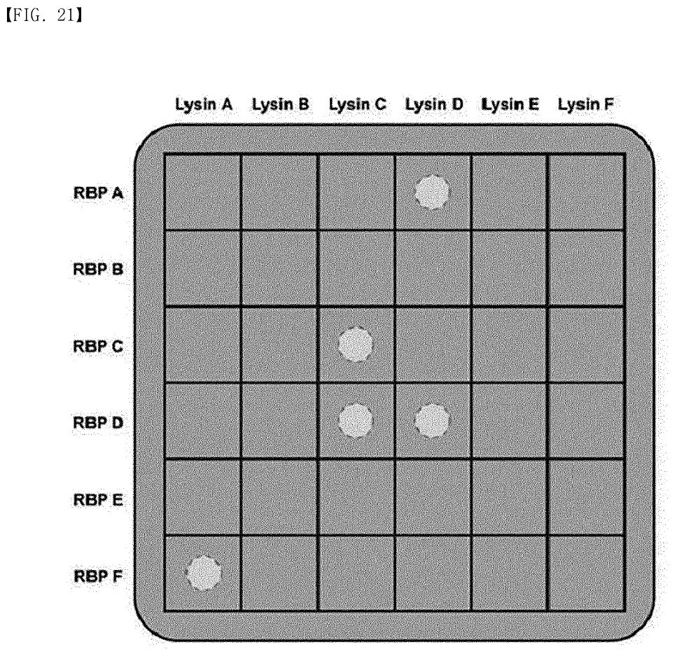

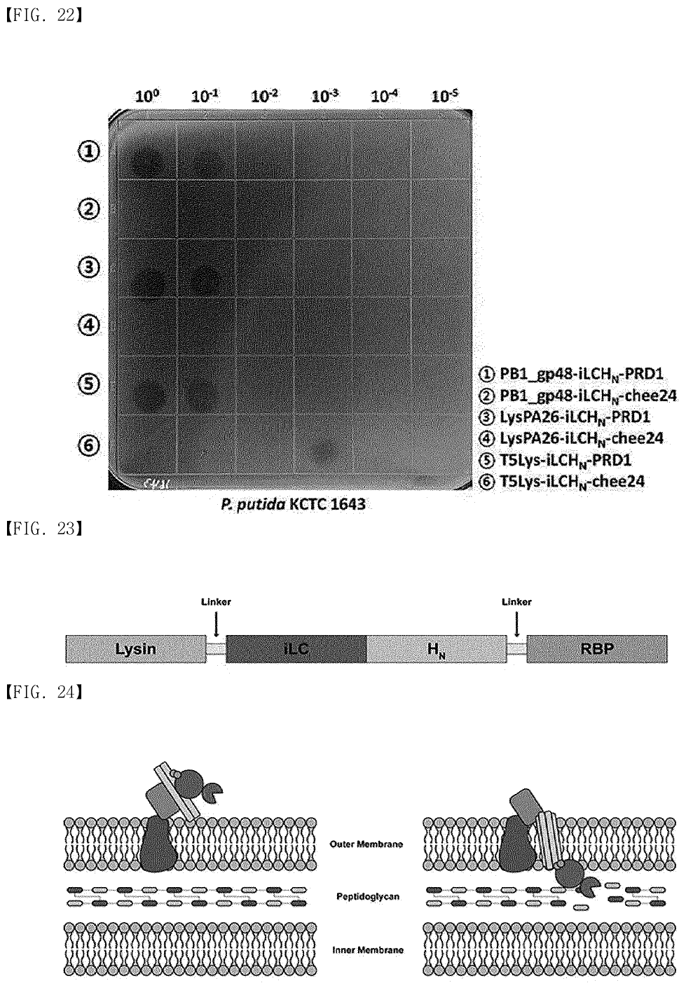

In the present invention, P. putida KCTC 1643 strain was treated with six types of translysins (PB1_gp48-iLCH_{N}-PRD1, PB1_gp48-iLCH_{N}-chee24, LysPA26-iLCH_{N}-PRD1, LysPA26-iLCH_{N}-chee24, T5Lys-iLCH_{N}-PRD1, T5Lys-iLCH_{N}-chee24), and the combinations exhibiting lytic activity were screened. The result showed that all translysins containing PRD1_04 RBP exhibited lytic activity against the target strain (FIG. 22).

The sequence additionally used to prepare the translysin is as follows:

T5Lys-Gp41.1N: SEQ ID NO: 27;
and
CfaC-T5 like chee24: SEQ ID NO: 28.

The translysin of the present invention is a protein complex in which an antibacterial protein endolysin derived from a bacteriophage and a receptor-binding protein (RBP) are linked to the translocation domain H_{N} of botulinum toxin, and was designed that RBP was disposed at the carboxyl terminus (C-terminal) and endolysin was disposed at the amino terminus (N-terminal) (FIG. 23) such that H_{N} was disposed between the RBP and the endolysin.

Based on this design, when translysin interacts with the target strain, the RBP at the carboxyl-terminus recognizes and attaches to the receptor located on the outer membrane, then H_{N} is incorporated into the cell membrane, and the endolysin at the amino terminal is close to peptidoglycan to induce lytic action and to kill cells (FIG. 24).

The translysin of the present invention was predicted to reach about 180 kDa in size. Considering the possibility of problems with yield and purity during the process of expressing and purifying translysin, a method of expressing the full-length protein and a method of separately expressing split protein fragments and then linking the fragments in vitro were performed respectively and compared. The result showed that the method of separately expressing the split protein fragments and then linking the same compensated for the drawbacks of the conventional method and was able to produce an intact full-length protein.

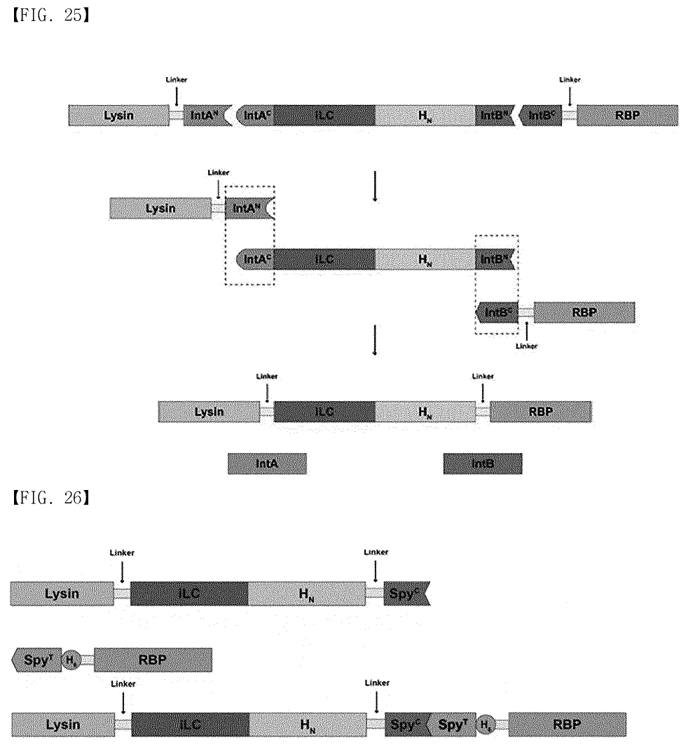

In the present invention, intein and SpyTag/Spycatcher are introduced as a means of binding respective fragments of endolysin, translocation domain, and RBP, and protein fragments are assembled into full-length proteins through protein trans-splicing and bioconjugation (FIGS. 25 and 26). In addition, novel antibacterial substances can be screened by combining various endolysins and RBPs using inteins and SpyTags/Spycatchers to construct a translysin library, and detecting the lytic activity of each translysin.

1.-10. (canceled)

11. An antibacterial composition comprising a protein complex comprising a botulinum toxin translocation domain and endolysin.

12. (canceled)

13. The antibacterial composition according to claim 11, wherein the botulinum toxin translocation domain is connected to a C-terminus of the endolysin.

14. The antibacterial composition according to claim 11 further comprising a receptor-binding protein.

15. The antibacterial composition according to claim 14, wherein the botulinum toxin translocation domain is connected to a C-terminus of the endolysin, and the receptor-binding protein is connected to a C-terminus of the botulinum toxin translocation domain.

16. The antibacterial composition according to claim 11, wherein the antibacterial composition has a lytic effect on Gram-negative bacteria.
